SEQUENCE LISTING (49 sequences)

SEQ ID NO: 1 - DNA, 642 bp, Artificial Sequence
  Mouse/human chimeric IgG1 Mouse light chain of DX-IgG
caaatcgtcttgtctcaatccccagctattttgtctgcttcccctggagagaaggtcaccatgacttgtagagcctcttcctctgtctcttacattcactggttccagcaaaagccaggttcctctccaaagccatggatctacgctacttccaacttggcttccggtgttccagttagattctctggttctggttccggtacctcctactctcttaccatctccagagttgaagccgaggacgctgctacttactactgtcagcaatggacttctaacccaccaactttcggtggtggtaccaaattggagattaagagaactgttgctgctccatccgttttcattttcccaccatccgacgaacaattgaagtctggtacagcttccgttgtttgtttgttgaacaacttctacccaagagaggctaaggttcagtggaaggttgacaacgctttgcaatccggtaactcccaagaatccgttactgagcaggattctaaggattccacttactccttgtcctccactttgactttgtccaaggctgattacgagaagcacaaggtttacgcttgtgaggttacacatcagggtttgtcctccccagttactaagtccttcaacagaggagagtgttaa

SEQ ID NO: 2 - DNA, 1356 bp, Artificial Sequence
  Mouse/human chimeric IgG1 heavy chain of DX-IgG
caagtccagttgcaacagcctggtgccgagttggtcaagccaggtgcttctgttaagatgtcctgtaaggcttctggttacactttcacctcctacaacatgcactgggtcaagcaaactccaggtagaggtttggagtggattggtgccatctacccaggtaacggtgacacttcttacaaccaaaaattcaagggaaaggctactcttaccgctgataagtcctcttccaccgcctacatgcaattgtcttccttgacttctgaagattctgctgtttactactgtgctagatccacctactacggtggagactggtacttcaacgtttggggtgctggtaccactgtcaccgtttccgctgcttctactaagggaccatccgtttttccattggctccatcctctaagtctacttccggtggtactgctgctttgggatgtttggttaaggactacttcccagagcctgttactgtttcttggaactccggtgctttgacttctggtgttcacactttcccagctgttttgcaatcttccggtttgtactccttgtcctccgttgttactgttccatcctcttccttgggtactcagacttacatctgtaacgttaaccacaagccatccaacactaaggttgacaagaaggttgagccaaagtcctgtgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaggagcagtacaactccacttacagagttgtttccgttttgactgttttgcaccaggattggttgaacggaaaggagtacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaaaggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgcacaaccactacactcaaaagtccttgtctttgtccccaggtaagtaa

SEQ ID NO: 3 - DNA, 324 bp, Artificial Sequence
  Light constant region of human IgG1
agaactgttgctgctccatccgttttcattttcccaccatccgacgaacaattgaagtctggtacagcttccgttgtttgtttgttgaacaacttctacccaagagaggctaaggttcagtggaaggttgacaacgctttgcaatccggtaactcccaagaatccgttactgagcaggattctaaggattccacttactccttgtcctccactttgactttgtccaaggctgattacgagaagcacaaggtttacgcttgtgaggttacacatcagggtttgtcctccccagttactaagtccttcaacagaggagagtgttaa

SEQ ID NO: 4 - DNA, 990 bp, Artificial Sequence
  Heavy constant region of human IgG1
tctactaagggaccatccgtttttccattggctccatcctctaagtctacttccggtggtactgctgctttgggatgtttggttaaggactacttcccagagcctgttactgtttcttggaactccggtgctttgacttctggtgttcacactttcccagctgttttgcaatcttccggtttgtactccttgtcctccgttgttactgttccatcctcttccttgggtactcagacttacatctgtaacgttaaccacaagccatccaacactaaggttgacaagaaggttgagccaaagtcctgtgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaggagcagtacaactccacttacagagttgtttccgttttgactgttttgcaccaggattggttgaacggaaaggagtacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaaaggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgcacaaccactacactcaaaagtccttgtctttgtccccaggtaagtaa

SEQ ID NO: 5 - DNA, 45 bp, Artificial Sequence
  Mouse light chain variable region overlapping oligonucleotide CD20LF1
aggagtcgtattcaaatcgtcttgtctcaatccccagctattttg

SEQ ID NO: 6 - DNA, 45 bp, Artificial Sequence
  Mouse light chain variable region overlapping oligonucleotide CD20LF2
tctgcttcccctggagagaaggtcaccatgacttgtagagcctct

SEQ ID NO: 7 - DNA, 45 bp, Artificial Sequence
  Mouse light chain variable region overlapping oligonucleotide CD20LF3
tcctctgtctcttacattcactggttccagcaaaagccaggttcc

SEQ ID NO: 8 - DNA, 45 bp, Artificial Sequence
  Mouse light chain variable region overlapping oligonucleotide CD20LF4
tctccaaagccatggatctacgctacttccaacttggcttccggt

SEQ ID NO: 9 - DNA, 45 bp, Artificial Sequence
  Mouse light chain variable region overlapping oligonucleotide CD20LF5
gttccagttagattctctggttctggttccggtacctcctactct

SEQ ID NO: 10 - DNA, 45 bp, Artificial Sequence
  Mouse light chain variable region overlapping oligonucleotide CD20LF5
cttaccatctccagagttgaagccgaggacgctgctacttactac

SEQ ID NO: 11 - DNA, 45 bp, Artificial Sequence
  Mouse light chain variable region overlapping oligonucleotide CD20LF7
tgtcagcaatggacttctaacccaccaactttcggtggtggtacc

SEQ ID NO: 12 - DNA, 36 bp, Artificial Sequence
  Mouse light chain variable region plus human light chain constant region overlapping oligonucleotide CD20LF8
aaattggagattaagagaactgttgctgctccatcc

SEQ ID NO: 13 - DNA, 45 bp, Artificial Sequence
  Mouse light chain variable region overlapping oligonucleotide CD20LR1
caacagttctcttaatctccaatttggtaccaccaccgaaagttg

SEQ ID NO: 14 - DNA, 45 bp, Artificial Sequence
  Mouse light chain variable region overlapping oligonucleotide CD20LR2
gtgggttagaagtccattgctgacagtagtaagtagcagcgtcct

SEQ ID NO: 15 - DNA, 45 bp, Artificial Sequence
  Mouse light chain variable region overlapping oligonucleotide CD20LR3
cggcttcaactctggagatggtaagagagtaggaggtaccggaac

SEQ ID NO: 16 - DNA, 44 bp, Artificial Sequence
  Mouse light chain variable region overlapping oligonucleotide CD20LR3
agaaccagagaatctaactggaacaccggaagccaagttggaag

SEQ ID NO: 17 - DNA, 45 bp, Artificial Sequence
  Mouse light chain variable region overlapping oligonucleotide CD20LR5
tagcgtagatccatggctttggagaggaacctggcttttgctgga

SEQ ID NO: 18 - DNA, 44 bp, Artificial Sequence
  Mouse light chain variable region overlapping oligonucleotide CD20LR6
ccagtgaatgtaagagacagaggaagaggctctacaagtcatgg

SEQ ID NO: 19 - DNA, 45 bp, Artificial Sequence
  Mouse light chain variable region overlapping oligonucleotide CD20LR7
tgaccttctctccaggggaagcagacaaaatagctggggattgag

SEQ ID NO: 20 - DNA, 21 bp, Artificial Sequence
  5′MlyI primer CD20L/up
aggagtcgtattcaaatcgtc

SEQ ID NO: 21 - DNA, 21 bp, Artificial Sequence
  3′ variable/5′constant primer LfusionRTVAAPS/up
agaactgttgctgctccatcc

SEQ ID NO: 22 - DNA, 20 bp, Artificial Sequence
  3′ constant region primer LfusionRTVAAPS/lp
ggatggagcagcaacagttc

SEQ ID NO: 23 - DNA, 30 bp, Artificial Sequence
  3′ primer CD20L/lp
ctggtaccttaacactctcctctgttgaag

SEQ ID NO: 24 - DNA, 45 bp, Artificial Sequence
  Mouse heavy chain variable region overlapping oligonucleotide CD20HF1
aggagtcgtattcaagtccagttgcaacagcctggtgccgagttg

SEQ ID NO: 25 - DNA, 45 bp, Artificial Sequence
  Mouse heavy chain variable region overlapping oligonucleotide CD20HF2
gtcaagccaggtgcttctgttaagatgtcctgtaaggcttctggt

SEQ ID NO: 26 - DNA, 45 bp, Artificial Sequence
  Mouse heavy chain variable region overlapping oligonucleotide CD20HF3
tacactttcacctcctacaacatgcactgggtcaagcaaactcca

SEQ ID NO: 27 - DNA, 45 bp, Artificial Sequence
  Mouse heavy chain variable region overlapping oligonucleotide CD20HF4
ggtagaggtttggagtggattggtgccatctacccaggtaacggt

SEQ ID NO: 28 - DNA, 45 bp, Artificial Sequence
  Mouse heavy chain variable region overlapping oligonucleotide CD20HF5
gacacttcttacaaccaaaaattcaagggaaaggctactcttacc

SEQ ID NO: 29 - DNA, 45 bp, Artificial Sequence
  Mouse heavy chain variable region overlapping oligonucleotide CD20HF6
gctgataagtcctcttccaccgcctacatgcaattgtcttccttg

SEQ ID NO: 30 - DNA, 45 bp, Artificial Sequence
  Mouse heavy chain variable region overlapping oligonucleotide CD20HF7
acttctgaagactctgctgtttactactgtgctagatccacctac

SEQ ID NO: 31 - DNA, 45 bp, Artificial Sequence
  Mouse heavy chain variable region overlapping oligonucleotide CD20HF8
tacggtggagactggtacttcaacgtttggggtgctggtaccact

SEQ ID NO: 32 - DNA, 36 bp, Artificial Sequence
  Mouse heavy chain variable region plus human heavy chain constant region overlapping oligonucleotide CD20HF9
gtcaccgtttccgctgcttctactaagggaccatcc

SEQ ID NO: 33 - DNA, 45 bp, Artificial Sequence
  Mouse heavy chain variable region plus human heavy chain constant region overlapping oligonucleotide CD20HR1
tagtagaagcagcggaaacggtgacagtggtaccagcaccccaaa

SEQ ID NO: 34 - DNA, 44 bp, Artificial Sequence
  Mouse heavy chain variable region overlapping oligonucleotide CD20HR2
cgttgaagtaccagtctccaccgtagtaggtggatctagcacag

SEQ ID NO: 35 - DNA, 45 bp, Artificial Sequence
  Mouse heavy chain variable region overlapping oligonucleotide CD20HR3
agtaaacagcagagtcttcagaagtcaaggaagacaattgcatgt

SEQ ID NO: 36 - DNA, 45 bp, Artificial Sequence
  Mouse heavy chain variable region overlapping oligonucleotide CD20HR4
aggcggtggaagaggacttatcagcggtaagagtagcctttccct

SEQ ID NO: 37 - DNA, 45 bp, Artificial Sequence
  Mouse heavy chain variable region overlapping oligonucleotide CD20HR5
tgaatttttggttgtaagaagtgtcaccgttacctgggtagatgg

SEQ ID NO: 38 - DNA, 45 bp, Artificial Sequence
  Mouse heavy chain variable region overlapping oligonucleotide CD20HR6
caccaatccactccaaacctctacctggagtttgcttgacccagt

SEQ ID NO: 39 - DNA, 45 bp, Artificial Sequence
  Mouse heavy chain variable region overlapping oligonucleotide CD20HR7
gcatgttgtaggaggtgaaagtgtaaccagaagccttacaggaca

SEQ ID NO: 40 - DNA, 45 bp, Artificial Sequence
  Mouse heavy chain variable region overlapping oligonucleotide CD20HR8
tcttaacagaagcacctggcttgaccaactcggcaccaggctgtt

SEQ ID NO: 41 - DNA, 21 bp, Artificial Sequence
  5′MlyI primer CD20H/up
aggagtcgtattcaagtccag

SEQ ID NO: 42 - DNA, 21 bp, Artificial Sequence
  5′ variable/constant primer HchainASTKGPs/up
gcttctactaagggaccatcc

SEQ ID NO: 43 - DNA, 21 bp, Artificial Sequence
  3′ variable/constant primer HchainASTKGPs/lp
ggatggtcccttagtagaagc

SEQ ID NO: 44 - DNA, 28 bp, Artificial Sequence
  3′ constant region primer HFckpn1/lp
ctggtattacttacctggggacaaagac

SEQ ID NO: 45 - DNA, 105 bp, Artificial Sequence
  Kar2 signal sequence with EcoRI
gaattcgaaacgatgctgtcgttaaaaccatcttggctgactttggcggcattaatgtatgccatgctattggtcgtagtgccatttgctaaacctgttagagct

SEQ ID NO: 46 - DNA, 70 bp, Artificial Sequence
  Overlapping oligonucleotide P.BiPss/UP1-EcoRI
aattcgaaacgatgctgtctttgaagccatcttggcttactttggctgctttgatgtacgctatgctttt

SEQ ID NO: 47 - DNA, 41 bp, Artificial Sequence
  Overlapping oligonucleotide P.BiPss/LP1
ccaaagtaagccaagatggcttcaaagacagcatcgtttcg

SEQ ID NO: 48 - DNA, 34 bp, Artificial Sequence
  Overlapping oligonucleotide P.BiPss/UP2
ggttgttgttccatttgctaagccagttagagct

SEQ ID NO: 49 - DNA, 59 bp, Artificial Sequence
  Overlapping oligonucleotide P.BiPss/LP2
agctctaactggcttagcaaatggaacaacaaccaaaagcatagcgtacatcaaagcag

(19) United States
(12) Patent Application Publication
(10) Pub. No.: US 2009/0136525 A1
(43) Pub. Date: May 28, 2009

(54) Immunoglobulins Comprising Predominantly a Glcnacman3Glcnac2 Glycoform

(76) Inventors: Tillman Gerngross, Hanover, NH (US); Stefan Wildt, Lebanon, NH (US); Huijuan Li, Hanover, NH (US)

Correspondence Address: MERCK AND CO., INC, P O BOX 2000, RAHWAY, NJ, 07065-0907, US

(21) Appl. No.: 11/990,722

(22) PCT Filed: Sep. 1, 2006
(86) PCT No.: PCT/US2006/034465

§ 371 Date: Feb. 20, 2008

Related U.S. Application Data
(60) Provisional application No. 60/714,109, filed on Sep. 2, 2005.
(60) Provisional application No. 60/714,108, filed on Sep. 2, 2005.

(51) Int. Cl. A61K 39/395; C12N 5/10; C12P 21/00; A61P 37/00

(52) U.S. Cl. 424/178.1; 435/326; 435/69.6

(57) ABSTRACT

Compositions and methods for producing compositions comprising immunoglobulins or immunoglobulin fragments having an N-linked glycosylation pattern consisting predominantly of the GlCNAcMan₃GlcNAc₂ N-glycan structure are disclosed. The GlCNAcMan₃GlcNAc₂ N-glycan structure effects an increase in binding to the FcγRiπ receptors and a decrease in binding to the FcγRH receptors.

### BACKGROUND OF THE INVENTION

(1) Field of the Invention

The present invention relates to compositions and methods for producing compositions comprising immunoglobulins or immunoglobulin fragments having an N-linked glycosylation pattern consisting of GlcNAcMan₃GlcNAc₂ as the predominant N-glycan. The GlcNAcMan₃GlcNAc₂ N-glycan structure has a modulatory effect on specific effector functions of the immunoglobulin.

(2) Description of Related Art

Glycoproteins mediate many essential functions in humans and other mammals, including catalysis, signaling, cell-cell communication, and molecular recognition and association. Glycoproteins make up the majority of non-cytosolic proteins in eukaryotic organisms (Lis and Sharon, 1993, Eur. J. Biochem. 218:1-27). Many glycoproteins have been exploited for therapeutic purposes, and during the last two decades, recombinant versions of naturally-occurring glycoproteins have been a major part of the biotechnology industry. Examples of recombinant glycosylated proteins used as therapeutics include erythropoietin (EPO), therapeutic monoclonal antibodies (mAbs), tissue plasminogen activator (tPA), interferon-γ (IFN-γ ), granulocyte-macrophage colony stimulating factor (GM-CSF), and human chorionic gonadotrophin (hCH) (Cumming et al., 1991, Glycobiology 1:115-130). Variations in glycosylation patterns of recombinantly produced glycoproteins have recently been the topic of much attention in the scientific community as recombinant proteins produced as potential prophylactics and therapeutics approach the clinic.

Antibodies or immunoglobulins are glycoproteins that play a central role in the humoral immune response. Antibodies may be viewed as adaptor molecules that provide a link between humoral and cellular defense mechanisms. Antigen-specific recognition by antibodies results in the formation of immune complexes that may activate multiple effector mechanisms, resulting in the removal and destruction of the complex. Within the general class of immunoglobulins (Ig), five classes (isotypes) of antibodies—IgM, IgD, IgG, IgA, and IgE—an be distinguished biochemically as well as functionally, while more subtle differences confined to the variable region account for the specificity of antigen binding. Amongst these five classes of immunoglobulins, there are only two types of light chain, which are termed lambda (λ) and kappa (κ). No functional difference has been found between antibodies having λ or κ chains, and the ratio of the two types of light chains varies from species to species. There are five heavy chain classes or isotypes, and these determine the functional activity of an antibody molecule. Each immunoglobulin isotype has a particular function in immune responses and their distinctive functional properties are conferred by the carboxy-terminal part of the heavy chain, where it is not associated with the light chain. IgG is the most abundant immunoglobulin isotype in blood plasma, (See for example, Immunobiology, Janeway et al, 6th Edition, 2004, Garland Publishing, New York).

The IgG molecule comprises a Fab (fragment antigen binding) domain with constant and variable regions and an Fc (fragment crystallized) domain. The C_{H}2 domain of each heavy chain contains a single site for N-linked glycosylation at an asparagine residue linking an N-glycan to the immunoglobulins molecule, usually at asparagine residue 297 (Asn-297) (Kabat et al., Sequences of proteins of immunological interest, Fifth Ed., U.S. Department of Health and Human Services, NIH Publication No. 91-3242).

Analyses of the structural and functional aspects of the N-linked oligosaccharides are of biological interest for three main reasons: (1) the glycosylation of the C_{H}2 domain has been conserved throughout evolution, suggesting an important role for the oligosaccharides; (2) the immunoglobulin molecule serves as a model system for the analysis of oligosaccharide heterogeneity (Rademacher and Dwek, 1984; Rademacher et al., 1982); and (3) antibodies comprise dimeric associations of two heavy chains, which place two oligosaccharide units in direct contact with each other, so that the immunoglobulin molecule involves both specific protein-carbohydrate and carbohydrate-carbohydrate interactions.

It has been shown that different glycosylation patterns of immunoglobulins are associated with different biological properties (Jefferis and Lund, 1997, Antibody Eng. Chem. Immunol., 65: 111-128; Wright and Morrison, 1997, Trends Biotechnol., 15: 26-32). However, only a few specific glycoforms are known to confer desired biological functions. For example, an immunoglobulin composition having decreased fucosylation on N-linked glycans is reported to have enhanced binding to human FcγRIII and therefore enhanced antibody-dependent cellular cytotoxicity (ADCC) (Shields et al., 2002, J. Biol Chem, 277: 26733-26740; Shinkawa et al., 2003, J. Biol. Chem. 278: 3466-3473). And, compositions of fucosylated G2 (Gal2GlcNAc2-Man3GlcNAc2) IgG made in CHO cells reportedly increase complement-dependent cytotoxicity (CDC) activity to a greater extent than compositions of heterogenous antibodies (Raju, 2004, U.S. Published Patent Application No. 2004/0136986). It has also been suggested that an optimal antibody against tumors would be one that bound preferentially to activate Fc receptors (FcγRI, FcγRIIa, FcγRIII) and minimally to the inhibitory FcγRIIb receptor (Clynes et al., 2000, Nature, 6:443-446). Therefore, the ability to enrich for specific glycoforms on immunoglobulins glycoproteins is highly desirable.

In general, the glycosylation structures (oligosaccharides) on glycoprotein will vary depending upon the expression host and culturing conditions. Therapeutic proteins produced in non-human host cells are likely to contain non-human glycosylation which may elicit an immunogenic response in humans—for example, hypermannosylation in yeast (Ballou, 1990, Methods Enzymol. 185:440-470); α(1,3)-fucose and β(1,2)-xylose in plants, (Cabanes-Macheteau et al., 1999, Glycobiology, 9: 365-372); N-glycolylneuraminic acid in Chinese hamster ovary cells (Noguchi et al., 1995. J. Biochem. 117: 5-62); and, Galα-1,3Gal glycosylation in mice (Borrebaeck et al., 1993, Immun. Today, 14: 477-479). Furthermore, galactosylation can vary with cell culture conditions, which may render some immunoglobulin compositions immunogenic depending on their specific galactose pattern (Patel et al., 1992. Biochem J. 285: 839-845). The oligosaccharide structures of glycoproteins produced by non-human mammalian cells tend to be more closely related to those of human glycoproteins. Thus, most commercial immunoglobulins are produced in mammalian cells. However, mammalian cells have several important disadvantages as host cells for protein production. Besides being costly, processes for expressing proteins in mammalian cells produce heterogeneous populations of glycoforms, have low volumetric titers, and require both ongoing viral containment and significant time to generate stable cell lines.

It is understood that different glycoforms can profoundly affect the properties of a therapeutic glycoprotein, including pharmacokinetics, pharmacodynamics, receptor-interaction and tissue-specific targeting (Graddis et al., 2002, Curr Pharm Biotechnol. 3: 285-297). In particular, for immunoglobulins, the oligosaccharide structure can affect properties relevant to protease resistance, the serum half-life of the antibody mediated by the FcRn receptor, binding to the complement complex C1, which induces complement-dependent cytoxicity (CDC), and binding to FcγR receptors, which are responsible for modulating the antibody-dependent cell-mediated cytoxicity (ADCC) pathway, phagocytosis and antibody feedback. (Nose and Wigzell, 1983; Leatherbarrow and Dwek, 1983; Leatherbarrow et al., 1985; Walker et al., 1989; Carter et al., 1992, Proc. Natl. Acad. Sci. USA, 89: 4285-4289).

Because different glycoforms are associated with different biological properties, the ability to enrich for one or more specific glycoforms can be used to elucidate the relationship between a specific glycoform and a specific biological function. After a desired biological function is associated with a specific glycoform pattern, a glycoprotein composition enriched for the advantageous glycoform structures can be produced. Thus, the ability to produce glycoprotein compositions that are enriched for particular glycoforms is highly desirable.

### BRIEF SUMMARY OF THE INVENTION

The present invention provides compositions comprising a plurality of immunoglobulins or immunoglobulin fragments, each immunoglobulin or fragment comprising at least one N-glycan attached thereto wherein the composition thereby comprises a plurality of N-glycans in which the predominant N-glycan species consists essentially of GlcNAcMan₃GlcNAc₂. Thus, the present invention provides compositions comprising immunoglobulins or fragments having GlcNAcMan₃GlcNAc₂ as the predominant N-glycan.

In particular embodiments, greater than 20 mole percent of the plurality of N-glycans consist essentially of GlcNAcMan₃GlcNAc₂. In further still embodiments, greater than 50 mole percent of the plurality of N-glycans consists essentially of GlcNAcMan₃GlcNAc₂. In further still embodiments, greater than 75 mole percent of the plurality of N-glycans consists essentially of GlcNAcMan₃GlcNAc₂. In further still embodiments, greater than 90 percent of the plurality of N-glycans consists essentially of GlcNAcMan₃GlcNAc₂. In other embodiments, the GlcNAcMan₃GlcNAc₂ N-glycan structure is present at a level that is from about 5 mole percent to about 50 mole percent more than the next most predominant N-glycan structure of said plurality of N-glycans. Further provided are compositions comprising anti-CD20 antibodies having GlcNAcMan₃GlcNAc₂ as the predominant N-glycan.

The immunoglobulins or fragments comprising the compositions herein exhibit decreased binding affinity to FcγRIIa and/or FcγRIIb receptor and increased binding affinity to FcγRIIIa and/or FcγRIIIb receptor. Therefore, on one aspect, the present invention provides a composition comprising a plurality of immunoglobulins or fragments, each immunoglobulin or fragment comprising at least one N-glycan attached thereto, wherein the composition thereby comprises a plurality of N-glycans in which the predominant N-glycan consists essentially of GlcNAcMan₃GlcNAc₂ wherein the immunoglobulins or fragments exhibit decreased binding affinity to FcγRIIa and/or FcγRIIb receptor. In another aspect, the present invention provides a composition comprising a plurality of immunoglobulins or fragments, each immunoglobulin or fragment comprising at least one N-glycan attached thereto wherein the composition thereby comprises a plurality of N-glycans in which the predominant N-glycan consists essentially of GlcNAcMan₃GlcNAc₂ wherein the immunoglobulins or fragments exhibit increased binding affinity to FcγRIIIa and/or FcγRIIIb receptor.

In a further aspect, the present invention provides a composition comprising a plurality of immunoglobulins or fragments, each immunoglobulin or fragment comprising at least one N-glycan attached thereto wherein the composition thereby comprises a plurality of N-glycans in which the predominant N-glycan consists essentially of GlcNAcMan₃GlcNAc₂ wherein the immunoglobulins or fragments are expected to exhibit increased antibody-dependent cellular cytoxicity (ADCC).

In further still aspects of the present invention, the above compositions of the present invention comprise immunoglobulins or fragments, which are essentially free of fucose or that lack fucose.

The composition of the present invention also comprises a pharmaceutical composition and a pharmaceutically acceptable carrier. The composition of the present invention also comprises a pharmaceutical composition of immunoglobulins or fragments which have been purified and incorporated into a diagnostic kit.

The present invention further provides methods for producing any one of the aforementioned compositions comprising a plurality of immunoglobulins or fragments, each immunoglobulin or fragment comprising at least one N-glycan attached thereto wherein the composition thereby comprises a plurality of N-glycans in which the predominant N-glycan consists essentially of GlcNAcMan₃GlcNAc₂. In one aspect, the method comprises the step of culturing a host cell, preferably a eukaryote host cell that has been genetically modified or selected to express the immunoglobulin or fragment. In particular aspects, the host cell comprises an exogenous gene encoding an immunoglobulin or fragment. Preferably, the host cell is genetically modified or engineered to produce glycoproteins, which are enriched for the GlcNAcMan₃GlcNAc₂ N-glycan. Therefore, in particular aspects, the host cells include one or more exogenous genes selected from the group consisting of α-1,2-manosidase, mannosidase II, UDP-GlcNAc transporter, and a GlcNAc transferase (GnT1). Preferably, the above host cells are also deficient for α-1,6-mannosyltransferase activity encoded by OCH1 and homologues. In further embodiments, the above host cells are also deficient for mannosylphosphorylation activity and in further still embodiments, the above host cells are also deficient in β-mannosylation activity. Thus, the present invention provides a method for producing a composition comprising a plurality of immunoglobulins or fragments, each immunoglobulin or fragment comprising at least one N-glycan attached thereto wherein the composition thereby comprises a plurality of N-glycans in which the predominant N-glycan consists essentially of GlcNAcMan₃GlcNAc₂ comprising (a) providing the eukaryote host cell above; (b) growing the eukaryote host cell in a culture medium for a time sufficient for the eukaryote host cell to produce the immunoglobulins or fragments; and, (c) isolating the immunoglobulins or fragments to produce the composition.

In a preferred aspect, the host cell is a lower eukaryote. Lower eukaryote cells include yeast, fungi, collar-flagellates, microsporidia, alveolates (for example, dinoflagellates), stramenopiles (e.g, brown algae, protozoa), rhodophyta (for example, red algae), plants (for example, green algae, plant cells, moss) and other protists. Yeast and fungi include, but are not limited to, Pichia sp., such as Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, and Pichia methanolica; Saccharomyces sp., such as Saccharomyces cerevisiae; Hansenula polymorpha, Kluyveromyces sp., such as Kluyveromyces lactis; Candida albicans, Aspergillus nidulans, Aspergillus niger, Aspergillus oryzae, Trichoderma reesei, Chrysosporium lucknowense, Fusarium sp., such as Fusarium gramineum, Fusarium venenatum, Physcomitrella patens, and Neurospora crassa. Preferred lower eukaryotes of the invention include but are not limited to Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzue, Trichoderma reseei, Chrysosporium lucknowense, Fusarium sp. Fusarium gramineum, Fusarium venenatum, and Neurospora crassa.

Therefore, the present invention further provides a method for producing any one of the aforementioned compositions comprising a plurality of immunoglobulins or fragments, each immunoglobulin or fragment comprising at least one N-glycan attached thereto wherein the composition thereby comprises a plurality of N-glycans in which the predominant N-glycan consists essentially of GlcNAcMan₃GlcNAc₂ in a lower eukaryote host cell. In particular aspects, the lower eukaryote host cell comprises an exogenous gene encoding the immunoglobulin or fragment and the host cell has been genetically modified or engineered to produce glycoproteins, which are enriched for the GlcNAcMan₃GlcNAc₂ N-glycan. Therefore, in particular aspects, the lower eukaryote host cells include one or more exogenous genes selected from the group consisting of α-1,2-manosidase, mannosidase II, GlcNAc transferase (GnT1), and UDP-GlcNAc transporter. Preferably, the above lower eukaryote host cells include each of the aforementioned exogenous genes. Preferably, the above lower eukaryote host cells are also deficient for α-1,6-mannosyltransferase activity encoded by the gene OCH1p or homologues thereof. In further embodiments, the above lower eukaryote host cells are also deficient for mannosylphosphorylation activity (deletion or disruption of the PNO1 and MNN4b genes) and in further still embodiments, the above eukaryote host cells are also deficient in β-mannosylation activity (deletion or disruption of one or more of the genes involved in β-mannosylation. Thus, the present invention provides a method for producing a composition comprising a plurality of immunoglobulins or fragments, each immunoglobulin or fragment comprising at least one N-glycan attached thereto wherein the composition thereby comprises a plurality of N-glycans in which the predominant N-glycan consists essentially of GlcNAcMan₃GlcNAc₂ comprising (a) providing the lower eukaryote host cell above; (b) growing the lower eukaryote host cell in a culture medium for a time sufficient for the lower eukaryote host cell to produce the immunoglobulins or fragments; and, (c) isolating the immunoglobulins or fragments to produce the composition.

The present invention further provides methods for increasing binding of an immunoglobulin or fragment to FcγRIIIa and/or FcγRIIIb receptors and decreasing binding of the immunoglobulin to FcγRIIa and/or FcγRIIb receptors or to increase ADCC by producing the immunoglobulin in one of the aforementioned host cells that has been engineered or selected to express the immunoglobulin in which GlcNAcMan₃GlcNAc₂ is the predominant N-glycan.

Unless otherwise defined herein, scientific and technical terms and phrases used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of biochemistry, enzymology, molecular and cellular biology, microbiology, genetics, and protein and nucleic acid chemistry and hybridization described herein are those well known and commonly used in the art.

### Definitions

The following terms, unless otherwise indicated, shall be understood to have the following meanings.

As used herein, the terms “antibody,” “immunoglobulin,” “immunoglobulins” and “immunoglobulin molecule” are used interchangeably. Each immunoglobulin molecule has a unique structure that allows it to bind its specific antigen, but all immunoglobulins have the same overall structure as described herein. The basic immunoglobulin structural unit is known to comprise a tetramer of subunits. Each tetramer has two identical pairs of polypeptide chains, each pair having one “light” chain (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Light chains are classified as either kappa or lambda. Heavy chains are classified as gamma, mu, alpha, delta, or epsilon, and define the antibody's isotype as IgG, IgM, IgA, IgD and IgE, respectively.

The light and heavy chains are subdivided into variable regions and constant regions (See generally, Fundamental Immunology (Paul, W., ed., 2nd ed. Raven Press, N.Y., 1989), Ch. 7. The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. The terms include naturally occurring forms, as well as fragments and derivatives. Included within the scope of the term are classes of immunoglobulins (Igs), namely, IgG, IgA, IgE, IgM, and IgD. Also included within the scope of the terms are the subtypes of IgGs, namely, IgG1, IgG2, IgG3 and IgG4. The term is used in the broadest sense and includes single monoclonal antibodies (including agonist and antagonist antibodies) as well as antibody compositions which will bind to multiple epitopes or antigens. The terms specifically cover monoclonal antibodies (including full length monoclonal antibodies), polyclonal antibodies, multispecific antibodies (for example, bispecific antibodies), and antibody fragments so long as they contain or are modified to contain at least the portion of the C_{H}2 domain of the heavy chain immunoglobulin constant region which comprises an N-linked glycosylation site of the C_{H}2 domain, or a variant thereof. Included within the terms are molecules comprising only the Fc region, such as immunoadhesins (U.S. Published Patent Application No. 20040136986), Fc fusions, and antibody-like molecules. Alternatively, these terms can refer to an antibody fragment of at least the Fab region that at least contains an N-linked glycosylation site.

The term “Fc” fragment refers to the ‘fragment crystallized’ C-terminal region of the antibody containing the C_{H}2 and C_{H}3 domains (FIG. 1). The term “Fab” fragment refers to the ‘fragment antigen binding’ region of the antibody containing the V_{H}, C_{H}1, V_{L} and C_{L} domains (See FIG. 1).

The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each mAb is directed against a single determinant on the antigen. In addition to their specificity, monoclonal antibodies are advantageous in that they can be synthesized by hybridoma culture, uncontaminated by other immunoglobulins. The term “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., (1975) Nature, 256:495, or may be made by recombinant DNA methods (See, for example, U.S. Pat. No. 4,816,567 to Cabilly et al.).

The term “fragments” within the scope of the terms “antibody” or “immunoglobulin” include those produced by digestion with various proteases, those produced by chemical cleavage and/or chemical dissociation and those produced recombinantly, so long as the fragment remains capable of specific binding to a target molecule. Among such fragments are Fc, Fab, Fab′, Fv, F(ab′)₂, and single chain Fv (scFv) fragments. Hereinafter, the term “immunoglobulin” also includes the term “fragments” as well.

Immunoglobulins further include immunoglobulins or fragments that have been modified in sequence but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (See, for example, Intracellular Antibodies: Research and Disease Applications, (Marasco, ed., Springer-Verlag New York, Inc., 1998).

As used herein, the term “consisting essentially of” will be understood to imply the inclusion of a stated integer or group of integers; while excluding modifications or other integers which would materially affect or alter the stated integer. With respect to species of N-glycans, the term “consisting essentially of” a stated N-glycan will be understood to include the N-glycan whether or not that N-glycan is fucosylated at the N-acetylglucosamine (GlcNAc) which is directly linked to the asparagine residue of the glycoprotein.

As used herein, the term “predominantly” or variations such as “the predominant” or “which is predominant” will be understood to mean the glycan species that has the highest mole percent (%) of total neutral N-glycans after the glycoprotein has been treated with PNGase and released glycans analyzed by mass spectroscopy, for example, MALDI-TOF MS or HPLC. In other words, the phrase “predominantly” is defined as an individual entity, such as a specific glycoform, is present in greater mole percent than any other individual entity. For example, if a composition consists of species A in 40 mole percent, species B in 35 mole percent and species C in 25 mole percent, the composition comprises predominantly species A, and species B would be the next most predominant species. Some host cells may produce compositions comprising neutral N-glycans and charged N-glycans such as mannosylphosphate. Therefore, a composition of glycoproteins can include a plurality of charged and uncharged or neutral N-glycans. In the present invention, it is within the context of the total plurality of neutral N-glycans in the composition in which GlcNAcMan₃GlcNAc₂. is the predominant N-glycan. Thus, as used herein, “predominant N-glycan” means that of the total plurality of neutral N-glycans in the composition, the predominant N-glycan is GlcNAcMan₃GlcNAc₂.

As used herein, the term “essentially free of” a particular sugar residue, such as fucose, or galactose and the like, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent. Thus, substantially all of the N-glycan structures in a glycoprotein composition according to the present invention are free of fucose, or galactose, or both.

As used herein, a glycoprotein composition “lacks” or “is lacking” a particular sugar residue, such as fucose or galactose, when no detectable amount of such sugar residue is present on the N-glycan structures at any time. For example, in preferred embodiments of the present invention, the glycoprotein compositions are produced by lower eukaryotic organisms, as defined above, including yeast (for example, Pichia sp.; Saccharomyces sp.; Kluyveromyces sp.; Aspergillus sp.), and will “lack fucose,” because the cells of these organisms do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

The interaction of antibodies and antibody-antigen complexes with cells of the immune system and the variety of responses, including antibody-dependent cell-mediated cytotoxicity (ADCC) and complement-dependent cytotoxicity (CDC), clearance of immunocomplexes (phagocytosis), antibody production by B cells and IgG serum half-life are defined respectively in the following: Daeron et al., 1997, Annu. Rev. Immunol. 15: 203-234; Ward and Ghetie, 1995, Therapeutic Immunol. 2:77-94; Cox and Greenberg, 2001, Semin. Immunol. 13: 339-345; Heyman, 2003, Immunol. Lett. 88:157-161; and Ravetch, 1997, Curr. Opin. Immunol. 9: 121-125.

### BRIEF DESCRIPTION OF THE DRAWINGS

FIG. 1 shows a schematic representation of an immunoglobulin molecule having a GlcNAcMan₃GlcNAc₂ N-glycan structure at Asn-297 of each C_{H}2 chain.

FIG. 2A shows a plasmid map of pDX343 encoding DX-IgG1 light chain in pCR2.1 TOPO vector.

FIG. 2B shows a plasmid map of pDX344 encoding Kar2 (Bip) signal sequence and DX-IgG1 light chain from pDX343

FIG. 2C shows a plasmid map of pDX360 encoding DX-IgG1 heavy chain in pCR2.1 TOPO vector.

FIG. 2D shows a plasmid map of pDX458 encoding the Kar2 SS and light chain from pDX344 in a pPICZA vector encoding AOX2 promoter.

FIG. 2E shows a plasmid map of pDX468 encoding Kar2 (Bip) signal sequence and DX-IgG1 heavy chain from DX-IgG1 from pDX360.

FIG. 2F shows a plasmid map of pDX478 encoding the Kar2 SS and DX-IgG1 heavy chain from pDX360 subcloned into pDX458 (Example 1).

FIG. 3 shows a MALDI-TOF spectra of sample F060708 isolated from strain YDX554 (DX-IgG1 having GlcNAcMan₃GlcNAc₂ as the predominant N-glycan expressed in strain YSH37).

FIG. 4 shows the results of an ELISA binding assay comparing the binding of DX-IgG1 (F060708) having GlcNAcMan₃GlcNAc₂ as the predominant N-glycan and RITUXIMAB to FcγRIIb.

FIG. 5A shows the results of an ELISA binding assay comparing the binding of DX-IgG1 (F060708) having GlcNAcMan₃GlcNAc₂ as the predominant N-glycan and RITUXIMAB to the FcγRIIIa-LF phenotype.

FIG. 5B shows the results of an ELISA binding assay comparing the binding of DX-IgG1 (F060708) having GlcNAcMan₃GlcNAc₂ as the predominant N-glycan and RITUXIMAB to the FcγRIIIa-LV phenotype.

### DETAILED DESCRIPTION OF THE INVENTION

The present invention provides compositions comprising a population of immunoglobulins or fragments having a plurality of N-glycans wherein the predominant N-glycan consists essentially of the structure GlcNAcMan₃GlcNAc₂. The GlcNAcMan₃GlcNAc₂ N-glycan structure can be specifically denoted as [(GlcNAcβ1,2-Manα1,3)(Manα1,6)Manβ1,4-GlcNAcβ1,4-GlcNAc].

The inventors show herein that the GlcNAcMan₃GlcNAc₂ N-glycan on immunoglobulins has an affect on particular antibody effector functions. For example, as shown herein, compositions comprising immunoglobulins wherein the predominant N-glycan is GlcNAcMan₃GlcNAc₂, the immunoglobulins have increased direct binding activity to the FcγRIIIa-LF and -LV receptors and decreased (or lack of) direct binding activity to the FcγRIIb receptor. In light of the above binding activities, immunoglobulins having GlcNAcMan₃GlcNAc₂ as the predominant N-glycan is expected to mediate other antibody effector functions, such as increasing ADCC activity or increasing antibody production by B cells while effecting a decrease in phagocytic activity. Therefore, a composition comprising a plurality of immunoglobulins having GlcNAcMan₃GlcNAc₂ as the predominant N-glycan, the immunoglobulins therein have increased binding activity to FcγRIII receptors and decreased binding activity to FcγRII receptors. Thus, the composition is expected to effect an increase in ADCC activity, increased antibody production by B cells, and decreased phagocytosis.

The present invention further provides methods for producing compositions comprising immunoglobulins having GlcNAcMan₃GlcNAc₂ as the predominant N-glycan. An advantage of producing immunoglobulins compositions having a predominant glycoform is that it avoids production of immunoglobulins having undesired glycoforms and/or production of heterogeneous mixtures of immunoglobulins, which may induce undesired effects and/or dilute the concentration of the more effective immunoglobulins glycoform(s). It is, therefore, contemplated that a pharmaceutical composition comprising immunoglobulins having GlcNAcMan₃GlcNAc₂ as the predominant N-glycan will may well be effective at lower doses, thus having higher efficacy or potency.

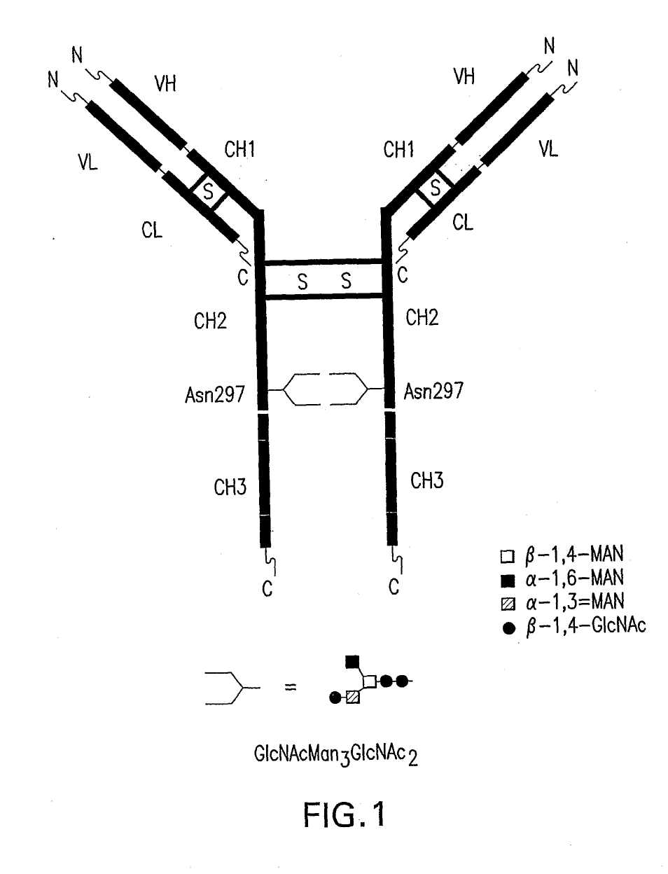

In one aspect, the immunoglobulin molecule comprising the composition has a GlcNAcMan₃GlcNAc₂ N-glycan structure at asparagine residue number 297 (Asn-297) of the C_{H}2 domain of the heavy chain on the Fc region in which the hydroxyl group of the terminal GlcNAc (N-acetyl-β-D-glucosamine) is covalently linked to the amide group of the asparagine at position 297. The Fc region mediates antibody effector function in an immunoglobulins molecule. Preferably, the GlcNAcMan₃GlcNAc₂ glycan structure is on each Asn-297 residue of each C_{H}2 region of a dimerized immunoglobulin (See FIG. 1). Therefore, provided are compositions of immunoglobulins wherein the predominant glycoform at Asn-297 is the GlcNAcMan₃GlcNAc₂ N-glycan structure. Alternatively, one or more other carbohydrate moieties found on an immunoglobulin molecule may be deleted and/or added to the molecule, thus adding or deleting the number of glycosylation sites on the immunoglobulin. Further, the position of the N-linked glycosylation site within the C_{H}2 region of the immunoglobulin molecule can be altered by introducing asparagines or other N-glycosylation sites at one or more other locations within the immunoglobulin molecule.

While Asn-297 is the N-glycosylation site typically found in murine and human IgG molecules (Kabat et al., Sequences of Proteins of Immunological Interest, 1991), the Asn-297 site is not the only site on the immunoglobulin molecule that can be glycosylated nor does the site have to be maintained for function. Using known methods for mutagenesis, a nucleic acid molecule encoding an immunoglobulin can be modified such that the nucleic acid sequence encoding the N-glycosylation site comprising Asn-297 is deleted or altered to be non-functional for N-glycosylation and a nucleic acid sequence encoding an N-glycosylation site is introduced at another position within the nucleic acid encoding the immunoglobulin molecule to produce an immunoglobulin having GlcNAcMan₃GlcNAc₂ as the predominant N-glycan at a non-native position. Additional nucleic acid sequences encoding N-glycosylation sites can be introduced into the nucleic acid above (or to a nucleic acid encoding the Asn-297 N-glycosylation site) to produce an immunoglobulin molecule having N-glycans in which GlcNAcMan₃GlcNAc₂ is the predominant N-glycan at more than one location within the molecule. However, it is preferred that the N-glycosylation sites are created within the C_{H}2 region of the immunoglobulin molecule. However, glycosylation of the Fab region of an immunoglobulin has been described in 30% of serum antibodies—commonly found at Asn-75 (Rademacher et al., 1986, Biochem. Soc. Symp., 51: 131-148). Therefore, glycosylation in the Fab region of an immunoglobulin molecule is an additional site that can be combined in conjunction with N-glycosylation in the Fc region, or alone.

In general, the composition comprises immunoglobulins wherein the predominant N-glycan is GlcNAcMan₃GlcNAc₂, which is present at a level that is at least about 5 mole percent more than the next predominant N-glycan structure of the recombinant immunoglobulin composition. In a preferred embodiment, the GlcNAcMan₃GlcNAc₂ N-glycan structure is present at a level of at least about 10 mole percent to about 25 mole percent more than the next predominant N-glycan structure of the recombinant immunoglobulin composition. In a more preferred embodiment, the GlcNAcMan₃GlcNAc₂ N-glycan structure is present at a level that is at least about 25 mole percent to about 50 mole percent more than the next predominant N-glycan structure of the recombinant immunoglobulin composition. In a more preferred embodiment, GlcNAcMan₃GlcNAc₂ N-glycan structure is present at a level that is greater than about 50 mole percent more than the next predominant N-glycan structure of the recombinant immunoglobulin composition. In more preferred embodiment, the GlcNAcMan₃GlcNAc₂ N-glycan structure is present at a level that is greater than about 75 mole percent more than the next predominant N-glycan structure of the recombinant immunoglobulin composition. In a most preferred embodiment, the GlcNAcMan₃GlcNAc₂ N-glycan structure is present at a level that is greater than about 90 mole percent more than the next predominant glycan structure of the recombinant immunoglobulin composition.

The immunoglobulin subclasses have been shown to have different binding affinities for Fc receptors (Huizinga et al., 1989, J. of Immunol., 142: 2359-2364). Each of the subclasses may offer particular advantages in different aspects of the present invention. Thus, provided are compositions comprising IgG1, IgG2, IgG3, IgG4, or mixtures thereof wherein the predominant N-glycan is GlcNAcMan₃GlcNAc₂. In further embodiments, compositions are provided wherein the immunoglobulin in which GlcNAcMan₃GlcNAc₂ is the predominant N-glycan is selected from the group consisting of IgA, IgD, IgE, IgM, and IgG. However, preferred immunoglobulins are human or humanized IgGs selected from the group consisting of the subtypes IgG1, IgG2, IgG3, and IgG4. More preferably, it is preferred that the immunoglobulin be an IgG1 subtype.

Preferably, the compositions comprise monoclonal immunoglobulins (antibodies) encoded by a nucleic acid, which when introduced into a host cell produces glycoproteins in which GlcNAcMan₃GlcNAc₂ is the predominant N-glycan. The monoclonal antibodies herein include for example “humanized antibodies”. Humanised antibodies can be obtained by complementary-determining region (CDR)-grafting (R. Kontermann & S. Duebel (2001) Recombinant antibodies—Laboratory Manuals. Springer Verlag ISBN 3-540-41354-5 and references therein). CDR-grafting consists of replacing the hypervariable loops of a human antibody with those of a monoclonal antibody (e.g. murine). Other approaches include ‘re-surfacing’ (Duebel & Kontermann (2001), Roguska et al. (1996) A comparison of two murine monoclonal antibodies humanized by CDR-grafting and variable domain resurfacing. Prot Eng. 9:895-904). Yet another approach to humanize antibodies consists of shuffling V- genes and selection on antigen. Shuffling of V-genes can be carried out, but is not restricted to, employing phage-display (Duebel & Kontermann (2001), Jespers et al. (1994) Guiding the selection of human antibodies from phage-display repertoires to a single epitope. Bio/Technol 12:899-903). Thus light chain variable domains of one origin can be spliced with a heavy chain constant domain from a different origin or vice versa, or a fusion of the variable or constant domain with heterologous protein, regardless of species of origin or immunoglobulin class or subclass designation, (See, for example, U.S. Pat. No. 4,816,567 to Cabilly et al.; Mage and Lamoyi, in Monoclonal Antibody Production Techniques and Applications, pp. 79-97 (Marcel Dekker, Inc., New York, 1987)).

The most common forms of humanized antibodies are human immunoglobulins in which residues from a CDR of the human immunoglobulin are replaced by residues from a CDR of a non-human species such as mouse, rat, or rabbit having the desired specificity, affinity, and capacity. In general, the humanized antibody will comprise substantially all of at least one, and typically two, variable domains, in which all or substantially all of the CDR regions correspond to those of a non-human immunoglobulin and all, or substantially all, of the framework (FR) regions are those of a human immunoglobulin consensus sequence. FR regions are the portions of the variable regions of an antibody that lie adjacent to or flank the CDRs. In general, these FR regions have more of a structural function that affects the conformation of the variable region and are less directly responsible for the specific binding of antigen to antibody, although, nonetheless, the FR regions can affect the interaction. The humanized antibody optimally also will comprise at least a portion of an immunoglobulin constant region (Fc), typically that of a human immunoglobulin. In some instances, FR residues of the human immunoglobulin are replaced by corresponding non-human residues. Furthermore, humanized antibodies can comprise residues, which are found neither in the recipient antibody nor in the imported CDR or FR sequences. These modifications are made to further refine and maximize antibody performance. For further details see Jones et al., 1986, Nature 321:522-524; Reichmann et al., 1988, Nature 332:323-327, and Presta, 1992, Curr. Op. Struct. Biol. 2:593-596.

The monoclonal antibodies herein further include “chimeric” antibodies (immunoglobulins) in which a portion of the heavy and/or light chain is identical with or homologous to corresponding sequences in antibodies derived from a first species or belonging to a particular antibody class or subclass, while the remainder of the chain(s) is identical with or homologous to corresponding sequences in antibodies derived from a different species or belonging to a different antibody class or subclass, as well as fragments of such antibodies, so long as they contain or are modified to contain at least one C_{H}2. “Humanized” forms of non-human (for example, murine) antibodies are specific recombinant immunoglobulins, immunoglobulin chains or fragments thereof (such as Fv, Fab, Fab′, F(ab′)₂, or other antigen-binding subsequences of antibodies) which contain sequences derived from human immunoglobulins. An Fv fragment of an antibody is the smallest unit of the antibody that retains the binding characteristics and specificity of the whole molecule. The Fv fragment is a noncovalently associated heterodimer of the variable domains of the antibody heavy chain and light chain. The F(ab)′₂ fragment is a fragment containing both arms of Fab fragments linked by the disulfide bridges. Example 1 illustrates the construction of expression vectors encoding a chimeric antibody comprising the mouse IgG1 variable domain against the antigen CD20 fused to the constant region of a human IgG1.

### Increased Binding of Immunoglobulins Having GlcNAcMan₃GlcNAc₂ as the Predominant N-Glycan to FcγRIII Receptors

The effector functions of immunoglobulin binding to FcγRIIa and/or FcγRIIIb receptors, such as activation of ADCC, are mediated by the Fc region of the immunoglobulin molecule. Different functions are mediated by the different domains in this region. FIGS. 6A and 6B show that a composition comprising an anti-CD20 antibody that has GlcNAMan₃ GlcNAc₂ as the predominant N-glycan (expressed in recombinant Pichia pastoris as described in Example 3) has increased binding to FcγRIIIa receptors compared to a composition in which the anti-CD20 antibodies (for example, RITUXIMAB) do not have GlcNAMan₃ GlcNAc₂ as the predominant N-glycan. Accordingly, the present invention provides immunoglobulin molecules and compositions in which the Fc region on the immunoglobulin molecule have GlcNAcMan₃GlcNAc₂ as the predominant N-glycan and wherein the immunoglobulin molecules have increased in binding to FcγRIIIa and/or FcγRIIIb receptors compared to immunoglobulins lacking GlcNAcMan₃GlcNAc₂ as the predominant N-glycan.

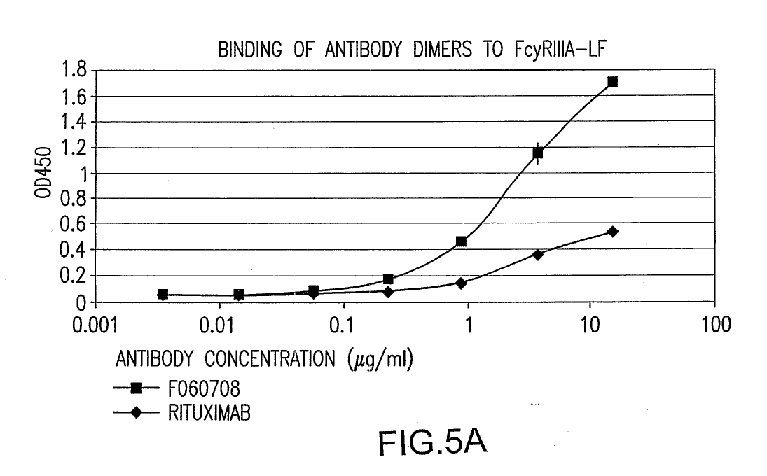

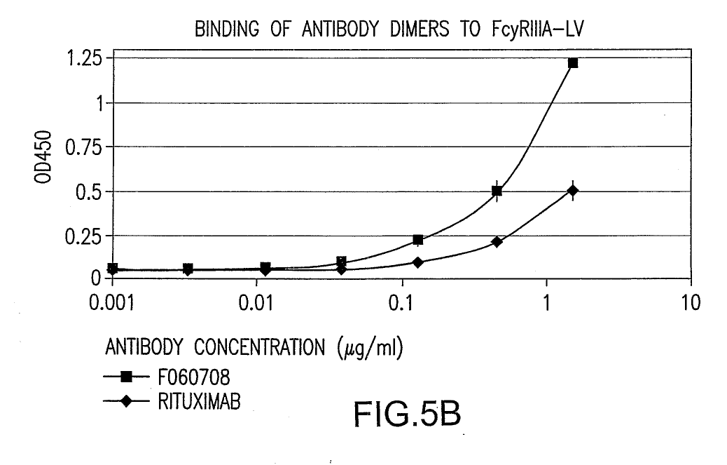

Interestingly, FcγRIIIa gene dimorphism results in two allotypes: FcγRIIIa-158V and FcγRIIIa-158F (Dall'Ozzo et al., 2004, Cancer Res. 64: 4664-4669). The genotype homozygous for FcγRIIIa-158V is associated with a higher clinical response to RITUXIMAB (Cartron et al., 2002, Blood, 99: 754-758). However, most of the population carries one FcγRIIIa-158F allele. In these heterozygous individuals, RITUXIMAB is less effective for induction of ADCC through FcγRIIIa binding. However, when a RITUXIMAB-like anti-CD20 antibody is expressed in a host cell that lacks fucosyltransferase activity, this antibody is equally effective for enhancing ADCC through both FcγRIIIa-158F and FcγRIIIa-158V (Niwa et al., 2004, Clin. Canc Res. 10: 6248-6255). The antibodies of certain preferred embodiments of the present invention are expressed in host cells that do not add fucose to N-glycans (for example, Pichia pastoris, a yeast host lacking the ability to add fucose). FIG. 5A shows that a composition comprising an anti-CD20 antibody that has GlcNAcMan₃GlcNAc₂ as the predominant N-glycan and expressed in recombinant Pichia pastoris as described in Example 3 has about a 3- to 4-fold increase in binding to the FcγRIIIa-LF receptor compared to RITUXIMAB, which does not have GlcNAcMan₃GlcNAc₂ as the predominant N-glycan, and FIG. 5B shows that the composition has about a 10-fold increase in binding to the FcγRIIIa-LV receptor compared to RITUXIMAB. Therefore, it is contemplated that anti-CD20 antibodies having GlcNAcMan₃GlcNAc₂ as the predominant N-glycan and that further lack fucose will have enhanced binding to FcγRIIIa-158F and may be especially useful for treating those individuals who have a reduced clinical response to RITUXIMAB.

Decreased Binding of Immunoglobulins having GlcNAcMan₃GlcNAc₂ as the Predominant N-Glycan to FcγRIIb Receptor

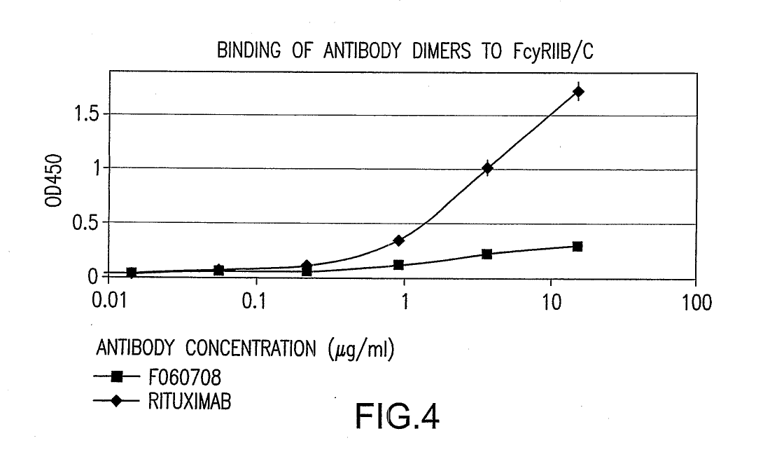

The effector functions of immunoglobulin molecules also include binding to the FcγRIIb receptors. Binding to the FcγRIIb such appears to result in decreased phagocytosis, decreased antibody production by B cells, and decreased ADCC activity. FIG. 4 shows that the immunoglobulins of the above composition comprising anti-CD20 antibodies that have GlcNAcMan₃GlcNAc₂ as the predominant N-glycan have decreased binding to FcγRIIb receptors compared to RITUXIMAB. Accordingly, the present invention provides immunoglobulin molecules and compositions in which the Fc region of the immunoglobulin molecule has GlcNAcMan₃GlcNAc₂ as the predominant N-glycan and which have decreased binding to FcγRIIb receptors.

### Increased Antibody-Dependent Cell-Mediated Cytoxicity

The increase in FcγRIIIa and/or FcγRIIIb binding of immunoglobulins having GlcNAcMan₃GlcNAc₂ as the predominant N-glycan may also confer an increase in FcγIII-mediated antibody-dependent cell-mediated cytoxicity (ADCC). It is well established that the FcγRIII (CD16) receptor is responsible for ADCC activity (Daeron et al., 1997, Annu. Rev. Immunol. 15: 203-234). The decrease in FcγRIIa and/or FcγRIIb binding of an immunoglobulins molecule or composition having GlcNAcMan₃GlcNAc₂ as the predominant N-glycan may also confer an increase in ADCC activity (See Clynes et al., 2000, supra). Therefore, immunoglobulin molecules having GlcNAcMan₃GlcNAc₂ as the predominant N-glycan or compositions comprising the immunoglobulins are expected to have increased ADCC activity.

### Decreased Phagocytosis (Clearance of Immunocomplexes by Macrophages)

In yet another embodiment, the decrease in FcγRIIa binding of immunoglobulins having GlcNAcMan₃GlcNAc₂ as the predominant N-glycan confers a decrease in FcγRIIa-mediated clearance of immune complexes (phagocytosis). It has been shown that the FcγRIIa (CD32) receptor is responsible for the clearance of immunocomplexes by macrophages (Cox and Greenberg, 2001, Semin. Immunol. 13: 339-345). Therefore, it is contemplated that immunoglobulins having GlcNAcMan₃GlcNAc₂ as the predominant N-glycan and compositions comprising the immunoglobulins may exhibit decreased phagocytosis.

### Increased Antibody Production by B Cells

Antibody engagement against tumors through the regulatory FcγR pathways has been shown (Clynes et al., 2000, Nature, 6: 443-446). Specifically, it is known when FcγRIIb is co-cross-linked with immunoreceptor tyrosine based activation motifs (ITAM)-containing receptors such as the B cell receptor (BCR), FcγRI, FcγRIII, and FcγRI, it inhibits ITAM-mediated signals (Vivier and Daeron, 1997, Immunol. Today, 18: 286-291). For example, the addition of FcγRII-specific antibodies blocks Fc binding to the FcγRIIb, resulting in augmented B cell proliferation (Wagle et al., 1999, J of Immunol. 162: 2732-2740). Accordingly, immunoglobulins having a GlcNAcMan₃GlcNAc₂ as the predominant N-glycan and compositions comprising the immunoglobulins are expected to mediate a decrease in FcγRIIb receptor binding resulting in the activation of B cells which in turn, catalyzes antibody production by plasma cells (Parker, D. C. 1993, Annu. Rev. Immunol. 11: 331-360).

### Other Immunological Activities

Altered surface expression of effector cell molecules on neutrophils has been shown to increase susceptibility to bacterial infections (Ohsaka et al., 1997, Br. J. Haematol. 98: 108-113). It has been further demonstrated that IgG binding to the FcγRIIIa effector cell receptors regulates expression of tumor necrosis factor alpha (TNF-α (Blom et al., 2004, Arthritis Rheum., 48: 1002-1014)). Furthermore, FcγR-induced TNF-α also increases the ability of neutrophils to bind and phagocytize IgG-coated erythrocytes (Capsoni et al., 1991, J. Clin. Lab Immunol. 34: 115-124). It is therefore contemplated that immunoglobulins having GlcNAcMan₃GlcNAc₂ as the predominant N-glycan and compositions comprising the immunoglobulins that show an increase in binding to FcγRIIIa receptor may also confer an increase in expression of TNF-α.

An increase in FcγRII and FcγRIII receptor activity has been shown to increase the secretion of lysosomal beta-glucuronidase as well as other lysosomal enzymes (Kavai et al., 1982, Adv. Exp Med. Biol. 141: 575-582; Ward and Ghetie, 1995, Therapeutic Immunol., 2: 77-94). Furthermore, an important step after the engagement of immunoreceptors by their ligands is their internalization and delivery to lysosomes (Bonnerot et al., 1998, EMBO J., 17: 4906-4916). It is therefore contemplated that immunoglobulins having GlcNAcMan₃GlcNAc₂ as the predominant N-glycan and compositions comprising the immunoglobulins that show an increase in binding to FcγRIIIa and/or FcγRIIIb receptor(s) may also confer an increase in the secretion of lysosomal enzymes.

Activation of more mature myeloid cells (for example mononuclear phagocytes, granulocytes and neutrophils) via binding to FcγRIIa results in enhanced superoxide production. Furthermore, the production of superoxide radicals by neutrophils is an important factor of the body defense system (Huizinga, et al., 1989, J Immunol., 142: 2365-2369). It is therefore contemplated that immunoglobulins having GlcNAcMan₃GlcNAc₂ as the predominant N-glycan and compositions comprising the immunoglobulins that show a decrease in binding to the FcγRIIa receptor may also confer a decrease in superoxide production.

Present exclusively on neutrophils, FcγRIIIb plays a predominant role in the assembly of immune complexes, and its aggregation activates phagocytosis, degranulation, and the respiratory burst leading to destruction of opsonized pathogens. Activation of neutrophils leads to secretion of a proteolytically cleaved soluble form of the receptor corresponding to its two extracellular domains. Soluble FcγRIIIb exerts regulatory functions by competitive inhibition of FcγR-dependent effector functions and via binding to the complement receptor CR3, leading to production of inflammatory mediators (Sautes-Fridman et al., 2003, ASHI Quarterly, 148-151). It is therefore contemplated that immunoglobulins having GlcNAcMan₃GlcNAc₂ as the predominant N-glycan and compositions comprising the immunoglobulins that show an increase in binding to the FcγRIIIb receptor may also facilitate assembly of immune complexes.

Production of Compositions Comprising Immunoglobulin Molecules having GlcNAcMan₃GlcNAc₂ as the Predominant N-Glycan

The immunoglobulins are produced in a host cell that has been genetically engineered to produce a composition of glycoproteins having GlcNAcMan₃GlcNAc₂ as the predominant N-glycan. In general, the recombinant host cells are transformed, preferably stably transformed, with one or more nucleic acids encoding the heavy and light chains of an immunoglobulin specific for a particular target antigen. In one embodiment, the nucleic acid encoding the heavy and light chains of the immunoglobulin are each separately synthesized using overlapping oligonucleotides and are each separately cloned into an expression vector (See Example 1) for expression in a host cell. In particular embodiments, the recombinant immunoglobulin encoded by the nucleic acid is a humanized immunoglobulin. Preferably, the recombinant host cells excrete the immunoglobulins into the culture medium used for culturing the recombinant cells. The recombinant host cells are then incubated under conditions suitable for producing the immunoglobulins, which will have GlcNAcMan₃GlcNAc₂ as the predominant N-glycan. The immunoglobulins are then separated from other components of the culture medium and resuspended in a suitable vehicle to make the compositions. While for many recombinant immunoglobulins the GlcNAcMan₃GlcNAc₂ will be linked to the nitrogen of the amide group of Asn-297, in particular embodiments, the site for the N-glycan linkage can be at an asparagine at a different site within the immunoglobulin molecule (other than Asn-297), or in combination with the N-glycosylation site in the Fab region.

The recombinant host cells may be a eukaryotic or prokaryotic host cell, such as an animal, plant, insect, bacterial cell, or the like which has been engineered or selected to produce immunoglobulin compositions having predominantly GlcNAcMan₃GlcNAc₂ N-glycan structures.

In a preferred embodiment, the immunoglobulin compositions in which GlcNAcMan₃GlcNAc₂ is the predominant N-glycan are produced in a lower eukaryote. Lower eukaryotic host cells do not normally produce glycoproteins which have GlcNAcMan₃GlcNAc₂ as the predominant N-glycan; however, lower eukaryotes can be genetically modified to produce glycoproteins which have GlcNAcMan₃GlcNAc₂ as the predominant N-glycan. Recombinant lower eukaryote cells genetically modified to produce glycoproteins having GlcNAcMan₃GlcNAc₂ as the predominant N-glycan are preferred over those mammalian cells which naturally produce glycoproteins having the GlcNAcMan₃GlcNAc₂ N-glycan but in low yield. Another advantage of using recombinant lower eukaryote host cells such as those described herein is that compositions of immunoglobulins can be reproducibly provided with GlcNAcMan₃GlcNAc₂ as the predominant N-glycan. A further still advantage is that lower eukaryotic cells can be grown in a defined culture medium that avoids the use of animal products such as calf serum.

Preferably, the recombinant host cell of the present invention is a lower eukaryotic host cell which has been genetically engineered or modified as described in WO 02/00879, WO 04/074498, WO 04/074499, Choi et al., 2003, PNAS, 100: 5022-5027; Hamilton et al., 2003, Nature, 301: 1244-1246 and Bobrowicz et al., 2004, Glycobiology, 14: 757-766, and Davidson et al, 2004 Glycobiology. 14(5):399-407. Lower eukaryote cells include yeast, fungi, collar-flagellates, microsporidia, alveolates (for example, dinoflagellates), stramenopiles (for example, brown algae, protozoa), rhodophyta (for example, red algae), plants (for example, green algae, plant cells, moss) and other protists. Yeast and fungi include, but are not limited to, Pichia sp., such as Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, and Pichia methanolica; Saccharomyces sp., such as Saccharomyces cerevisiae; Hansenula polymorpha, Kluyveromyces sp., such as Kluyveromyces lactis; Candida albicans, Aspergillus nidulans, Aspergillus niger, Aspergillus oryzae, Trichoderma reesei, Chrysosporium lucknowense, Fusarium sp., such as Fusarium gramineum, Fusarium venenatum, Physcomitrella patens, and Neurospora crassa. Preferred lower eukaryotes of the invention include but are not limited to Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzue, Trichoderma reseei, Chrysosporium lucknowense, Fusarium sp. Fusarium gramineum, Fusarium venenatum, and Neurospora crassa. A particularly preferred species is Pichia pastoris.

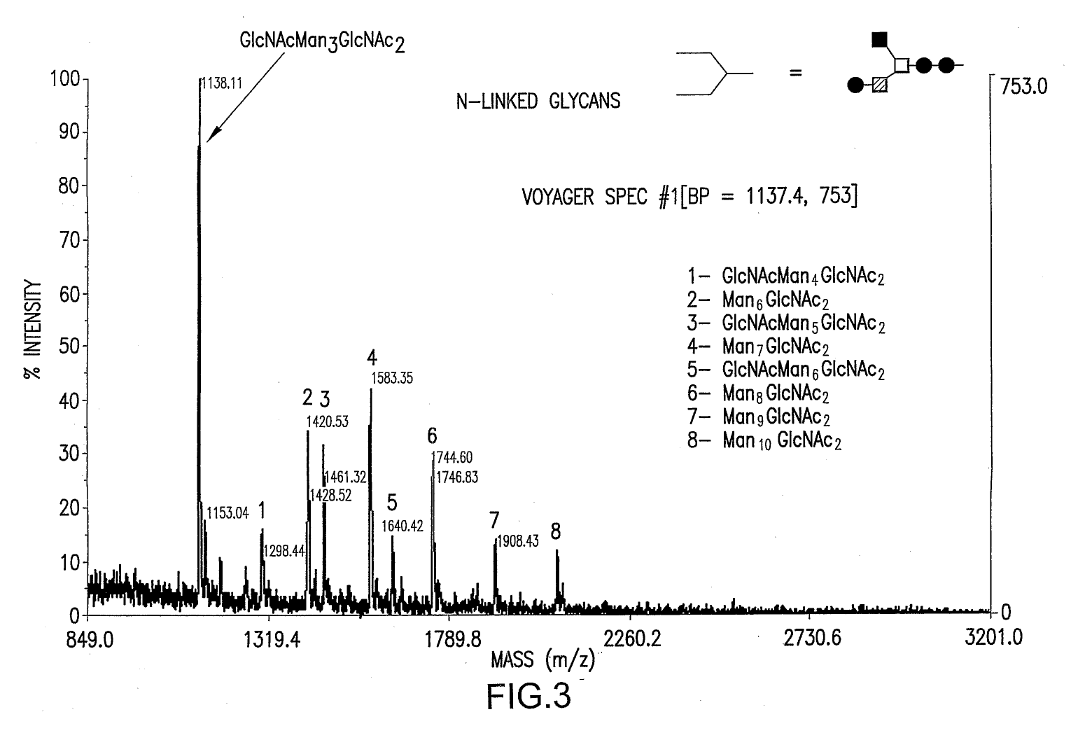

An embodiment for producing immunoglobulins having GlcNAcMan₃GlcNAc₂ as the predominant N-glycan is shown in Example 2. In Example 2, a vector encoding a chimeric immunoglobulin comprising the heavy and light chain variable regions of mouse IgG1 specific for CD20 linked to the heavy and light chain constant regions of human IgG1 was introduced into the recombinant yeast Pichia pastoris YSH37 strain (Hamilton et al., 2003, Science, 301: 1244-1246). The YSH37 recombinant yeast strain lacks endogenous α-1,6-mannosyltransferase activity (Och1p) and contains three heterologous genes: a gene encoding α-1,2-mannosidase (MnsIA), which is localized to the endoplasmic reticulum, and genes encoding UDP-N-acetylglucosamine (UDP-GlcNAc) transporter, β-1,2-N-acetylglucosaminyltransferase 1 (GlcNAc transferase 1 or GnT1), and mannosidase II (MnsII), all localized to the golgi. In general, the heterologous genes comprise synthetic fusions between fungal type II membrane proteins and catalytic domains from organisms other than Pichia pastoris. Because glycoproteins produced in the recombinant yeast strain have predominantly the GlcNAcMan₃GlcNAc₂ N-glycan structure, immunoglobulins such as the immunoglobulin of Example 2 that are produced in the recombinant yeast strain will have GlcNAcMan₃GlcNAc₂ as the predominant N-glycan. FIG. 3 shows that the anti-CD20 immunoglobulin produced in the YSH37 recombinant yeast strain had GlcNAcMan₃GlcNAc₂ as the predominant N-glycan. About 20% of the glycoforms consisted of GlcNAcMan₃GlcNAc₂ with a plurality of other glycoforms in lesser amounts.

In further embodiments, the above recombinant yeast strain includes deletions or disruptions of the PNO1 and MNN4b genes, which results in the elimination of mannosylphosphorylation (See, for example U.S. Published Pat. Application No. 20060160179). Mannosylphosphorylation results in production of N-glycans that are charged. This further genetic modification provides a recombinant yeast strain capable of producing immunoglobulin compositions in which GlcNAcMan₃GlcNAc₂ is the predominant N-glycan and wherein the immunoglobulins are free of mannosylphosphate (and thus net negative charge), which may confer aberrant immunogenic activities in humans. In other embodiments, the above recombinant yeast strain includes deletions or disruptions of one or more of the genes involved in β-mannosylation (See, WO2005106010 and related U.S. patent application Ser. No. 11/118,008). These further genetic modifications provide a recombinant yeast strain capable of producing immunoglobulin compositions in which GlcNAcMan₃GlcNAc₂ is the predominant N-glycan and wherein the immunoglobulins are free of β-mannosylation, which may confer aberrant immunogenic activities in humans. In further still embodiments, the above recombinant yeast strain includes deletions and disruptions of the PNO1 and MNN4b genes and one or more of the genes involved in β-mannosylation. These further genetic modifications provide a recombinant yeast strain capable of producing immunoglobulin compositions in which GlcNAcMan₃GlcNAc₂ is the predominant N-glycan and wherein the immunoglobulins are free of mannosylphosphorylation and β-mannosylation.

While recombinant yeast cells have been described for producing immunoglobulins having GlcNAcMan₃GlcNAc₂ as the predominant N-glycan, other protein expression host systems including animal, plant, insect, bacterial cells and the like can be used to produce immunoglobulin having GlcNAcMan₃GlcNAc₂ as the predominant N-glycan. Such protein expression host systems may be genetically engineered or modified or selected to express immunoglobulins having GlcNAcMan₃GlcNAc₂ as the predominant N-glycan or may naturally produce glycoproteins having GlcNAcMan₃GlcNAc₂ as the predominant N-glycan structure. Examples of engineered protein expression host systems producing a glycoprotein having a predominant glycoform include gene knockouts/mutations (Shields et al., 2002, JBC, 277: 26733-26740); genetic engineering in Chinese hamster ovary cells (Umaña et al., 1999, Nature Biotech., 17: 176-180) or a combination of both. Alternatively, certain cells naturally express a predominant glycoform—for example, chickens, humans and cows (Raju et al., 2000, Glycobiology, 10: 477-486). These cells can be modified to produce immunoglobulins having GlcNAcMan₃GlcNAc₂ as the predominant N-glycan. Thus, the expression of an immunoglobulin or composition having GlcNAcMan₃GlcNAc₂ as the predominant N-glycan can be obtained by one skilled in the art by selecting at least one of many expression host systems. Further expression host systems include CHO cells: WO9922764A1 and WO03035835A1; hybridroma cells: Trebak et al., 1999, J. Immunol. Methods, 230: 59-70; insect cells: Hsu et al., 1997, JBC, 272:9062-970, and plant cells: WO04074499A2.

### Purification of Immunoglobulins

Methods for the purification and isolation of immunoglobulins are known (See, for example, Kohler & Milstein, (1975) Nature 256:495; Brodeur et al., Monoclonal Antibody Production Techniques and Applications, pp. 51-63, Marcel Dekker, Inc., New York, (1987);. Goding, Monoclonal Antibodies: Principles and Practice, pp. 59-104 (Academic Press, 1986); and Jakobovits et al., 1993, Proc. Natl. Acad. Sci. USA 90:2551-255, and Jakobovits et al., 1993, Nature 362:255-258). In a further embodiment, antibodies or antibody fragments can be isolated from antibody phage libraries generated using the techniques described in McCafferty et al. (1990) Nature, 348:552-554 (1990), using the antigen of interest to select for a suitable antibody or antibody fragment.

Example 3 provides a method for isolating the immunoglobulin molecules having GlcNAcMan₃GlcNAc₂ as the predominant N-glycan, which have been produced in genetically modified yeast cells genetically modified to produce glycoproteins having GlcNAcMan₃GlcNAc₂ as the predominant N-glycan. The glycan analysis and distribution on the isolated immunoglobulin molecule can be determined by several mass spectroscopy methods known to one skilled in the art, including but not limited to, HPLC, NMR, LCMS, and MALDI-TOF MS. In a preferred embodiment, the glycan distribution is determined by MALDI-TOF MS analysis as disclosed in Example 5.

### Pharmaceutical Compositions

Immunoglobulins having GlcNAcMan₃GlcNAc₂ as the predominant N-glycan can be incorporated into pharmaceutical compositions wherein the immunoglobulin is an active therapeutic agent (See Remington's Pharmaceutical Science (15th ed., Mack Publishing Company, Easton, Pa., 1980). The preferred composition depends on the intended mode of administration and therapeutic application. The composition can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers and the like.

Pharmaceutical compositions for parenteral administration are sterile, substantially isotonic, pyrogen-free, sterile, and prepared in accordance with GMP of the U.S. Food and Drug Administration or similar body. The compositions can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. The compositions can be administered in the form of a depot injection or implant preparation which can be formulated in such a manner as to permit a sustained release of the active ingredient. Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (See Langer, Science 249, 1527 (1990) and Hanes, Advanced Drug Delivery Reviews 28, 97-119 (1997).

### Diagnostic Products

The immunoglobulin molecules having GlcNAcMan₃GlcNAc₂ as the predominant N-glycan can also be incorporated into a variety of diagnostic kits and other diagnostic products such as an array. Immunoglobulins are often provided prebound to a solid phase, such as to the wells of a microtiter dish. Kits also often contain reagents for detecting immunoglobulin binding, and labeling providing directions for use of the kit. Immunometric or sandwich assays are a preferred format for diagnostic kits (See U.S. Pat. Nos. 4,376,110, 4,486,530, 5,914,241, and 5,965,375). Antibody arrays are described for example in U.S. Pat. Nos. 5,922,615, 5,458,852, 6,019,944, and 6,143,576.

The immunoglobulin molecules of the present invention having GlcNAcMan₃GlcNAc₂ as the predominant N-glycan have many therapeutic applications for indications such as cancers, inflammatory diseases, infections, immune diseases, autoimmune diseases including idiopathic thrombocytopenic purpura, arthritis, systemic lupus erythrematosus, and autoimmune hemolytic anemia. Targets of interest include growth factor receptors (for example, FGFR, PDGFR, EGFR, NGFR, and VEGF) and their ligands. Other targets are G protein receptors and include substance K receptor, the angiotensin receptor, the α- and β-adrenergic receptors, the serotonin receptors, and PAF receptor (See, for example, Gilman, Ann. Rev. Biochem. 56:625-649 (1987). Other targets include ion channels (for example, calcium, sodium, potassium channels), muscarinic receptors, acetylcholine receptors, GABA receptors, glutamate receptors, and dopamine receptors (See Harpold, U.S. Pat. No. 5,401,629 and U.S. Pat. No. 5,436,128). Other targets are adhesion proteins such as integrins, selectins, and immunoglobulin superfamily members (See Springer, Nature 346:425-433 (1990). Osborn, Cell 62:3 (1990); Hynes, Cell 69:11 (1992)). Other targets are cytokines, such as interleukins IL-1 through IL-13, tumor necrosis factors α and β, interferons α, β and γ, tumor growth factor Beta (TGF-β), colony stimulating factor (CSF) and granulocyte monocyte colony stimulating factor (GMCSF). See Human Cytokines: Handbook for Basic & Clinical Research (Aggrawal et al. eds., Blackwell Scientific, Boston, Mass. 1991). Other targets are hormones, enzymes, and intracellular and intercellular messengers, such as, adenyl cyclase, guanyl cyclase, and phospholipase C. Other targets of interest are leukocyte antigens, such as CD20, and CD33. Drugs may also be targets of interest. Target molecules can be human, mammalian or bacterial. Other targets are antigens, such as proteins, glycoproteins and carbohydrates from microbial pathogens, both viral and bacterial, and tumors. Still other targets are described in U.S. Pat. No. 4,366,241.

The methods and techniques of the present invention are generally performed according to conventional methods well known in the art and as described in various general and more specific references that are cited and discussed throughout the present specification unless otherwise indicated. See, for example, Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999); Immunobiology, Janeway et al, 6th Edition, 2004, Garland Publishing, New York).

All publications, patents and other references mentioned herein are hereby incorporated by reference in their entireties.

The following examples are intended to promote a further understanding of the present invention.

### EXAMPLE 1

A vector encoding a chimeric anti-CD20 monoclonal antibody consisting of a light (L) chain fusion protein having the mouse light chain variable region fused to the human light chain constant region and a heavy (H) chain fusion protein consisting of the mouse variable heavy chain region fused to the human heavy chain constant region was constructed for producing a humanized anti-CD20 monoclonal antibody having GlcNAcMan₃GlcNAc₂ as the predominant N-glycan in recombinant Pichia pastoris, which had been genetically modified to produce glycoproteins having GlcNAcMan₃GlcNAc₂ as the predominant N-glycan.

Cloning of nucleic acid encoding the chimeric anti-CD20 monoclonal antibody, DX-IgG1, for expression in Pichia pastoris was essentially as follows. The light and heavy chains of DX-IgG1 chimeric antibody consists of mouse variable regions and human constant regions. The nucleotide sequence encoding the mouse/human chimeric light chain is shown in SEQ ID NO: 1 and the nucleotide sequence encoding the mouse/human chimeric heavy chain shown in SEQ ID NO: 2. The heavy and light chain encoding nucleic acids are synthesized using overlapping oligonucleotides purchased from Integrated DNA Technologies (IDT).

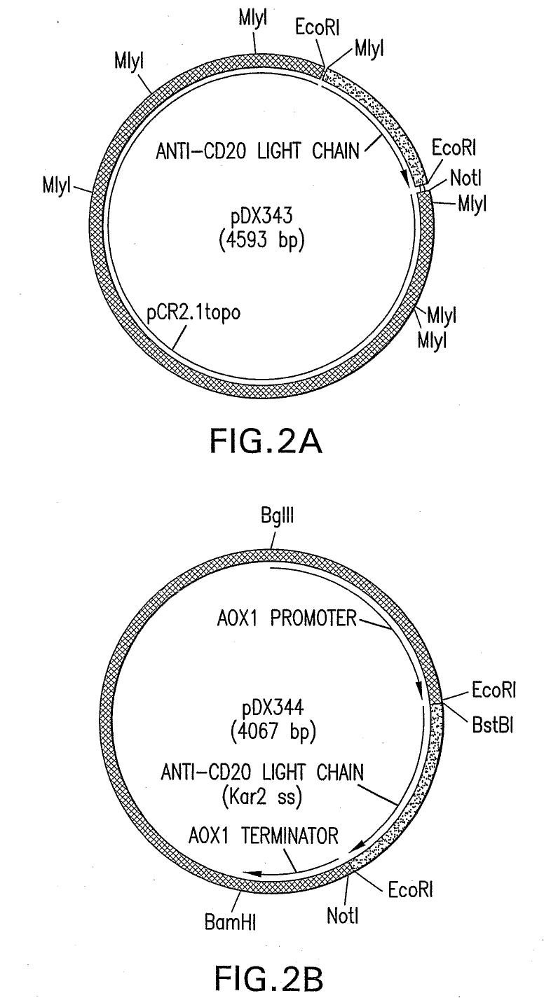

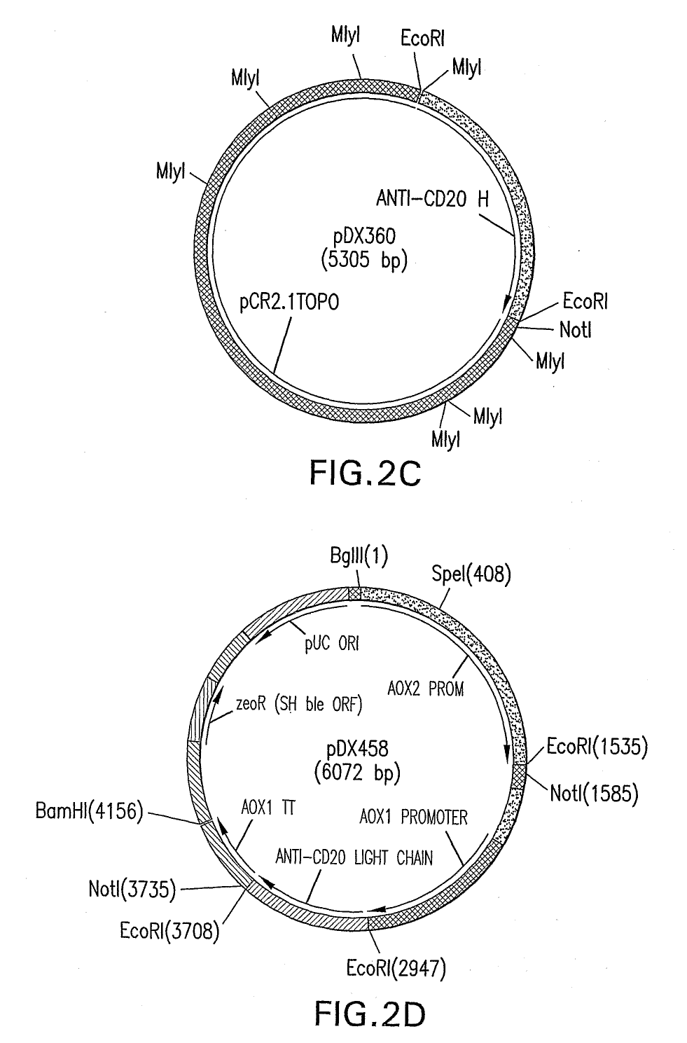

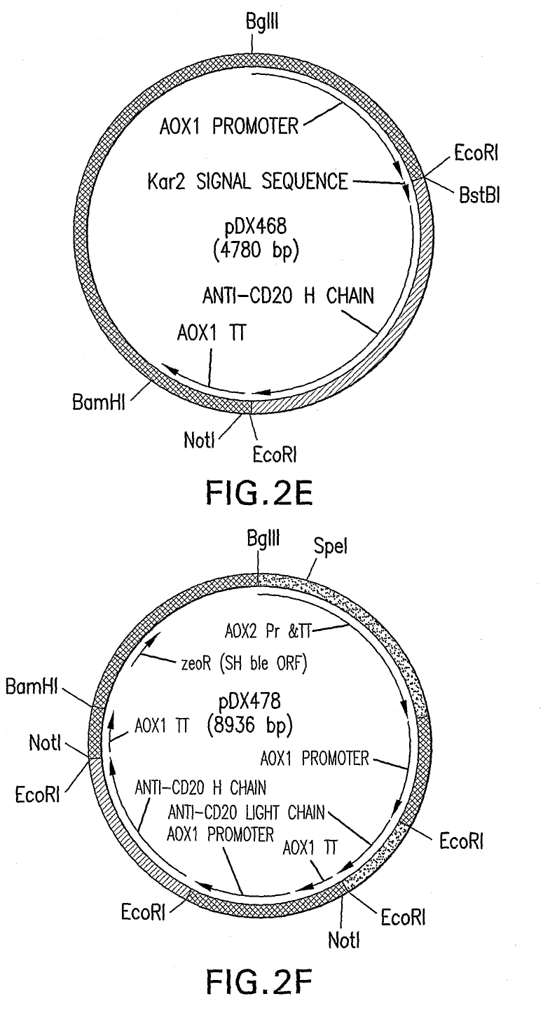

For synthesizing a nucleic acid encoding the light chain variable region, 15 overlapping oligonucleotides (SEQ ID NOs: 5-19) were purchased and annealed using EX TAQ (Takada) in a PCR reaction to produce a nucleic acid encoding the light chain variable region having a 5′ MlyI site. This light chain variable encoding nucleic acid was then joined in frame with a nucleic acid encoding the light chain constant region (SEQ ID NO: 3) (Gene Art, Toronto, Canada) by overlapping PCR using the 5′ MlyI primer CD20L/up (SEQ ID NO: 20), the 3′ variable/5′ constant primer LfusionRTVAAPS/up (SEQ ID NO: 21), the 3′ constant region primer Lfusion RTVAAPS/lp (SEQ ID NO: 22) and 3′ CD20L/lp (SEQ ID NO: 23). The final MlyI nucleic acid encoding the chimeric mouse-human light chain fragment (which included 5′AG base pairs) was then inserted into pCR2.1 TOPO vector (Invitrogen Corporation, Carlsbad, Calif.) resulting in pDX343 (FIG. 2A).

For the heavy chain, 17 overlapping oligonucleotides (SEQ ID NOs: 24-40) corresponding to the nucleic acid sequence encoding the mouse heavy chain variable region were purchased from IDT and annealed using EX TAQ. This nucleic acid encoding the mouse heavy chain variable fragment was then joined in frame with a nucleic acid encoding the human heavy chain constant region (SEQ ID NO: 4) (Gene Art) by overlapping PCR using the 5′ MlyI primer CD20H/up (SEQ ID NO: 41), the 5′ variable/constant primer HchainASTKGPS/up (SEQ ID NO: 42), the 3′ variable/constant primer HchainASTKGPS/lp (SEQ ID NO: 43), and the 3′ constant region primer HFckpn1/lp (SEQ ID NO: 44). The final MlyI nucleic acid encoding the chimeric mouse-human heavy chain fragment (which included 5′AG base pairs) was inserted into pCR2.1 TOPO vector resulting in pDX360 (FIG. 2C).

The nucleic acids encoding the full-length chimeric light chain and full-length chimeric heavy chain were isolated from the respective TOPO vectors as Mly1-Not1 nucleic acid fragments. These light chain and heavy chain encoding nucleic acid fragments were each then ligated to a Kar2 (Bip) signal sequence (SEQ ID NO: 45) using 4 overlapping oligonucleotides—P.BiPss/UP1-EcoRI, P.BiPss/LP1, P.BiPss/UP2 and P.BiP/LP2 (SEQ ID NOS: 46-49, respectively), and then ligated into the EcoRI-Not1 sites of pPICZA resulting in pDX344 carrying the Kar2-light chain and AOX1 transcription termination sequence (AOX1 terminator or TT) (FIG. 2B) and pDX468 carrying the Kar2-heavy chain (FIG. 2E).

A BglII-BamHI fragment from pDX344 was then subcloned into pBK85 containing the AOX2 promoter gene for chromosomal integration, resulting in pDX458 (FIG. 2D).

A BglII-BamHI fragment from pDX468 carrying the heavy chain was then subcloned into pDX458, resulting in pDX478 (FIG. 2F), which encodes both the full-length chimeric heavy and light chains of the anti-CD20 monoclonal antibody under control of the AOX1 promoter. The chimeric antibody encoded by the pDX478 was designated DX-IgG1. Plasmid pDX478 was then linearized with SpeI prior to transformation for integration into the AOX2 locus with transformants selected using Zeocin resistance (See Example 2).

RITUXIMAB/RITUXAN is an anti-CD20 mouse/human chimeric IgG1 purchased from Biogen-IDEC/Genentech, San Francisco, Calif.

PCR amplification. An Eppendorf Mastercycler (Westbury, N.Y.) was used for all PCR reactions. PCR reactions contained template DNA, 125 μM dNTPs, 0.2 μM each of forward and reverse primer, EX TAQ polymerase buffer (Takara Bio Inc., Shiga, Japan), and EX TAQ polymerase or pFU Turbo polymerase buffer (Stratagene) and pFU Turbo polymerase. The DNA fragments were amplified with 30 cycles of 15 seconds at 97° C., 15 seconds at 55° C., and 90 seconds at 72° C. with an initial denaturation step of two minutes at 97° C. and a final extension step of seven minutes at 72° C.

PCR samples were separated by agarose gel electrophoresis and the DNA bands are extracted and purified using a Gel Extraction Kit from Qiagen. All DNA purifications were eluted in 10 mM Tris, pH 8.0 except for the final PCR (overlap of all three fragments), which was eluted in deionized H₂O.

### EXAMPLE 2

This example shows a method for producing the chimeric humanized anti-CD20 monoclonal antibodies having GlcNAcMan₃GlcNAc₂ as the predominant N-glycan encoded by the pDX478 or pJC140 in recombinant yeast cells.

Transformation of IgG vectors into the Pichia pastoris strain YSH37 (Hamilton et al., 2003) was essentially as follows. The vector DNA of pDX478 was prepared by adding sodium acetate to a final concentration of 0.3 M. One hundred percent ice cold ethanol was then added to a final concentration of 70% to the DNA sample. The DNA was pelleted by centrifugation (12000 g×10 minutes) and washed twice with 70% ice cold ethanol. The DNA was dried and resuspended in 50 μL of 10 mM Tris, pH 8.0.

The yeast cells to be transformed were prepared by expanding a smaller culture in BMGY (buffered minimal glycerol: 100 mM potassium phosphate, pH 6.0; 1.34% yeast nitrogen base; 4×10−5% biotin; 1% glycerol) to an O.D. of about 2 to 6. The yeast cells were then made electrocompetent by washing 3 times in 1M sorbitol and resuspending in about 1 to 2 mL 1M sorbitol. Vector DNA (1 to 2 μg) was mixed with 100 μL of competent yeast and incubated on ice for 10 minutes. Yeast cells were then electroporated with a BTX Electrocell Manipulator 600 using the following parameters; 1.5 kV, 129 ohms, and 25 μF. One milliliter of YPDS (1% yeast extract, 2% peptone, 2% dextrose, 1M sorbitol) was added to the electroporated cells. Transformed yeast is subsequently plated on selective agar plates containing zeocin.

Culture conditions for IgG1 production in Pichia pastoris were essentially as follows. A single colony of the YSH37 strain described above transformed with pDX478 was inoculated into 10 mL of BMGY media (consisting of 1% yeast extract, 2% peptone, 100 mM potassium phosphate buffer (pH 6.0), 1.34% yeast nitrogen base, 4×10 5% biotin, and 1% glycerol) in a 50 ml Falcon Centrifuge tube. The culture was incubated while shaking at 24° C./170-190 rpm for 48 hours until the culture is saturated. 100 mL of BMGY is then added to a 500 ml baffled flask. The seed culture was then transferred into a baffled flask containing the 100 mL of BMGY media. This culture was incubated with shaking at 24° C. at 170 to 190 rpm for 24 hours. The contents of the flask were decanted into two 50 mL Falcon Centrifuge tubes and centrifuged at 3000 rpm for 10 minutes. The cell pellet was washed once with 20 mL of BMGY without glycerol, followed by gentle resuspension with 20 ml of BMMY (BMGY with 1% MeOH instead of 1% glycerol). The suspended cells were transferred into a 250 mL baffled flask. The culture was incubated with shaking at 24° C. at 170 to 190 rpm for 24 hours. The contents of the flask was then decanted into two 50 mL Falcon Centrifuge tubes and centrifuged at 3000 rpm for 10 minutes. The culture supernatant was analyzed by ELISA to determine approximate antibody titer prior to protein isolation as described in Example 6.

Quantification of antibody in culture supernatants was performed by enzyme linked immunosorbent assays (ELISAs). High binding microtiter plates (Costar) were coated with 24 μg of goat anti-human Fab (Biocarta, Inc, San Diego, Calif.) in 10 mL PBS, pH 7.4 and incubated over night at 4° C. Buffer was removed and blocking buffer (3% BSA in PBS), is added and then incubated for one hour at room temperature. Blocking buffer was removed and the plates washed 3 times with PBS. After the last wash, increasing volume amounts of antibody culture supernatant (0.4, 0.8, 1.5, 3.2, 6.25, 12.5, 25, and 50 μL) were added and the plates incubated for one hour at room temperature. Plates were then washed with PBS containing 0.05% Tween 20. After the last wash, anti-human Fc-HRP was added in a 1:2000 PBS solution, and then incubated for 1 hour at room temperature. Plates were then washed 4 times with PBS-Tween 20. Plates were analyzed using TMB substrate kit following manufacturer's instructions (Pierce Biotechnology).

Yeast strain DX554 was produced according to the method shown above for transforming pDX478 into recombinant yeast strain YSH37.

### EXAMPLE 3

Purification of the chimeric anti-CD20 monoclonal antibodies produced in Example 2 was essentially as follows. The antibodies produced by yeast cells transformed with pDX478 were designated DX-IgG1.

Antibodies were captured from the culture supernatant using a STREAMLINE Protein A column (Amersham Biosciences, Piscataway, N.J.). Antibodies were eluted in Tris-Glycine pH 3.5 and neutralized using IM Tris pH 8.0. Further purification was carried out using hydrophobic interaction chromatography (HIC). The specific type of HIC column depends on the antibody. For the DX-IgG1, a phenyl SEPHAROSE column (can also use octyl SEPHAROSE) was used with 20 mM Tris (7.0), 1M (NH₄)₂SO₄ buffer and eluted with a linear gradient buffer starting at 1M (NH₄)₂SO₄ and decreasing to 0M (NH₄)₂SO₄. The antibody fractions from the phenyl SEPHAROSE column were pooled and exchanged into 50 mM NaOAc/Tris pH 5.2 buffer for final purification through a cation exchange (SP SEPHAROSE Fast Flow) (GE Healthcare) column. Antibodies were eluted with a linear gradient using 50 mM Tris, 1M NaCl (pH 7.0). DX-IgG1 antibodies were isolated from the culture medium of cultures of DX554 grown according to Example 2.

The concentration of protein in the chromatography fractions was determined using a Bradford assay (Bradford, M. 1976, Anal. Biochem. (1976) 72, 248-254) using albumin as a standard (Pierce Chemical Company, Rockford, Ill.).

### EXAMPLE 4

Detection of purified antibodies by SDS-polyacrylamide gel electrophoresis was as follows.

Purified DX-IgG1 antibodies were mixed with an appropriate volume of sample loading buffer and subjected to sodium dodecyl sulfate-polyacrylamide gel electrophoresis (SDS-PAGE) with precast gels according to the manufacturer's instructions (NuPAGE bis-Tris Electrophoresis System; Invitrogen Corporation). The gel proteins were stained with Coomassie brilliant blue stain (Bio-Rad, Hercules, Calif.).

### EXAMPLE 5

Matrix Assisted Laser Desorption Ionization Time of Flight Mass Spectrometry (MALDI-TOF MS) was used to analyze the Asn-linked oligosaccharides on the DX-IgG1 antibodies having GlcNAcMan₃GlcNAc₂ as the predominant neutral N-glycan produced in Example 2.

The N-linked glycans were released from the antibodies using a modified procedure from Papac et al. (Glycobiology 8, 445-454 (1998). Briefly, an antibody sample was denatured and applied to a 96-well PVDF membrane plate. The sample was then reduced with dithiothreitol and carboxymethylated with iodoacetic acid. The wells were then blocked with polyvinylpyridine. The antibody sample was then deglycosylated by incubation with 1 mU of N-glycanase (EMD Biosciences, La Jolla, Calif.) in 30 μL of 10 mM NH₄HCO₃ (pH 8.3) for 16 hours at 37° C. The solution containing the released glycans was then removed by centrifugation through the PVDF membrane and evaporated to dryness. The dried glycans from each well were dissolved in 15 μL of water and 0.5 μL is spotted onto stainless-steel MALDI sample plates and mixed with 0.5 μL of S-DHB matrix (9 mg/mL of dihydroxybenzoic acid/1 mg/mL of 5-methoxy-salicylic acid in 1:1 water/acetonitrile/0.1% trifluoroacetic acid) and allowed to dry. Ions were generated by irradiation with a pulsed nitrogen laser (337 nm) with a 4-ns pulse time. The instrument was operated in the delayed extraction mode with a 125-ns delay and an accelerating voltage of 20 kV. The grid voltage was 93.00%, guide wire voltage was 0.1%, the internal pressure is less than 5×10⁷ torr (1 torr=133 Pa), and the low mass gate was 850 Da. Spectra were generated from the sum of 100-200 laser pulses and acquired with a 500-MHz digitizer. Man₅GlcNAc₂ (Mr 1257 [M⁺Na]⁺) oligosaccharide was used as an external molecular weight standard. All spectra were generated with the instrument in the positive-ion mode.

FIG. 3 shows a MALDI-TOF MS spectra of the composition from fermentation No. F060708 comprising DX-IgG1 antibodies, which had been produced by YDX554 cells according to the protocol in Example 2. FIG. 3 shows that the predominant N-glycan structure in the composition is GlcNAcMan₃GlcNAc₂. However, as shown in FIG. 3, the composition includes other N-glycan structures as well. These N-glycans include GlcNAcMan₄GlcNAc₂, Man₆GlcNAc₂; GlcNAcMan₅GlcNAc₂, Man₇GlcNAc₂, GlcNAcMan₆GlcNAc₂, Man₈GlcNAc₂, Man₉GlcNAc₂, and Man₁₀GlcNAc₂.

To determine the relative amounts of the various neutral N-glycan structures, an HPLC was performed and the area under the peaks corresponding to each of the above N-glycan structures was determined from the HPLC scan measuring intensity verses retention time. The HPLC was a fast amino-silica glycans separation using a PREVAIL Carbohydrate ES 5 μm 250 mm×4.6 mm (Cat # 35101; Alltech Associates, Avondale, Pa.). The sample volume was 45 μL and the solvent was acetonitrile and LSS (50 mM NH₄ Formate pH 4.4). The flow Rate was 1.0 mL/min and the column temperature was 30° C. The Gradient was as follows: time 0, 80% acetonitrile:20% LSS; time 50, 40% acetonitrile, 60% LSS; time 55, 30% acetonitrile, 70% LSS; time 60, 80% acetonitrile, 20% LSS; and, time 70, 80% acetonitrile, 20% LSS. The results of the HPLC are shown in Table 1. The HPLC analysis showed that the predominant N-glycan structure GlcNAcMan₃GlcNAc₂ was found to comprise about 20% of the total neutral N-glycan structures.

TABLE 1

| Retention Time | | Concentration | |
|---|---|---|---|
| (minutes) | Area | (%) | Structure |
| 27.04 | 3168279 | 23.138 | GlcNAcMan₃GlcNAc₂ |
| 27.79 | 92781 | 0.678 | |
| 29.64 | 1026177 | 7.494 | GlcNAcMan₄GlcNAc₂ |
| 30.25 | 336324 | 2.456 | Man₅GlcNAc₂ |
| 32.26 | 1231613 | 8.995 | GlcNAcMan₅GlcNAc₂ |
| 32.69 | 1768694 | 12.917 | Man₆GlcNAc₂ |
| 34.72 | 2214086 | 16.170 | Man₇GlcNAc₂ |
| 36.51 | 1684304 | 12.301 | Man₈GlcNAc₂ |
| 37.17 | 858505 | 6.270 | Man₉GlcNAc₂ |
| 38.67 | 1141546 | 8.337 | Man₁₀GlcNAc₂ |
| 39.90 | 170462 | 1.245 | Man₁₁GlcNAc₂ |

### EXAMPLE 6

Fc Receptor binding assays for FcγRIIb, FcγRIIIa and FcγRIIIb were carried out according to the protocols described in Shields et al., 2001, J. Biol. Chem, 276: 6591-6604.

For the FcγRIIb binding assay, FcγRIIb fusion proteins at one μg/mL in PBS, pH 7.4, were coated onto ELISA plates (Nalge-Nunc, Naperville, Ill.) for 48 hours at 4° C. Plates were blocked with 3% bovine serum albumin (BSA) in PBS at 25° C. for one hour. DX-IgG1 or RITUXIMAB dimeric complexes were prepared in 1% BSA in PBS by mixing 2:1 molar amounts of DX-IgG1 or RITUXIMAB and HRP-conjugated F(Ab′)2 anti-F(Ab′)2 at 25° C. for one hour. Dimeric complexes were then diluted serially at 1:2 in 1% BSA/PBS and coated onto the plate for one hour at 25° C. The substrate used was 3,3′,5,5′-tetramethylbenzidine (TMB) (Vector Laboratories, Inc., Burlingame, Calif.). Absorbance at 450 nm was read following instructions of the manufacturer (Vector Laboratories, Inc.).

For the FcγRIIIa-LF and FcγRIIIa-LV binding assays, FcγRIIIa-LF or -LV fusion proteins at 0.8 μg/mL and 0.4 μg/mL, respectively, in PBS, pH 7.4, were coated onto ELISA plates (Nalge-Nunc, Naperville, Ill.) for 48 hours at 4° C. Plates were blocked with 3% BSA in PBS at 25° C. for one hour. DX-IgG1 or RITUXIMAB dimeric complexes were prepared in 1% BSA in PBS by mixing 2:1 molar amounts of DX-IgG1 or RITUXIMAB and HRP-conjugated F(Ab′)2 anti-F(Ab′)2 at 25° C. for one hour. Dimeric complexes were then diluted serially at 1:2 in 1% BSA/PBS and coated onto the plate for one hour at 25° C. The substrate used was 3,3′,5,5′-tetramethylbenzidine (TMB) (Vector Laboratories, Inc.). Absorbance at 450 nm was read following instructions of the manufacturer (Vector Laboratories, Inc.).

Binding results obtained in accordance with the above methods for FcγRIIb and FcγRIIIa-LF and -LV with glycoproteins produced from YDX554 (strain YSH37 expressing DX-IgG1) are shown in FIGS. 5 and 6A and 6B, respectively.

FIG. 4 shows that the above composition comprising anti-CD20 antibodies that have GlcNAcMan₃GlcNAc₂ as the predominant N-glycan has decreased binding to FcγRIIb receptors compared to RITUXIMAB.

FIG. 5A shows that a composition comprising an anti-CD20 antibody that have GlcNAcMan₃GlcNAc₂ as the predominant N-glycan and expressed in recombinant Pichia pastoris as described in Example 3 has about a 3-4-fold increase in binding to the FcγRIIIa-LF receptor compared to RITUXIMAB, which does not have GlcNAcMan₃GlcNAc₂ as the predominant N-glycan.

FIG. 5B shows that the composition has about a 10-fold increase in binding to the FcγRIIIa-LV receptor compared to RITUXIMAB. Therefore, antibody compositions produced from the cell line genetically engineered to produce glycoproteins comprising GlcNAcMan₃GlcNAc₂ as the predominant N-glycan had decreased binding to FcγRIIb and increased binding to FcγRIIIa.

### DESCRIPTION OF THE SEQUENCES

SEQ ID NO: 1 encodes the nucleotide sequence of the DX-IgG1 light chain.

SEQ ID NO: 2 encodes the nucleotide sequence of the DX-IgG1 heavy chain.

SEQ ID NO: 3 encodes the nucleotide sequence of the human constant region of an IgG1 light chain.

SEQ ID NO: 4 encodes the nucleotide sequence of the human constant region of an IgG1 heavy chain.

SEQ ID NO: 5 to 19 encode 15 overlapping oligonucleotides used to synthesize by polymerase chain reaction (PCR) the murine light chain variable region of DX-IgG1.

SEQ ID NO: 20 to 23 encode four oligonucleotide primers used to ligate the DX-IgG1 murine light chain variable region to a human light chain constant region.

SEQ ID NO: 24 to 40 encode 17 overlapping oligonucleotides used to synthesize by PCR the murine heavy chain variable region of DX-IgG1.

SEQ ID NO: 41 to 44 encode four oligonucleotide primers used to ligate the DX-IgG1 murine heavy chain variable region to a human heavy chain constant region.

SEQ ID NO: 45 encodes the nucleotide sequence encoding the Kar2 (Bip) signal sequence with an N-terminal EcoRI site.

SEQ ID NO: 46 to 49 encode four oligonucleotide primers used to ligate the Kar2 signal sequence to the light and heavy chains of DX-IgG1.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1: A composition comprising a plurality of immunoglobulins or fragments, each immunoglobulin or fragment comprising at least one N-glycan attached thereto wherein the composition thereby comprises a plurality of N-glycans in which the predominant N-glycan consists essentially of GlcNAcMan₃GlcNAc₂.

2: The composition of claim 1, wherein greater than 50 mole percent of said plurality of N-glycans consists essentially of GlcNAcMan₃GlcNAc₂.

3: The composition of claim 1, wherein greater than 75 mole percent of said plurality of N-glycans consists essentially of GlcNAcMan₃GlcNAc₂.

4: The composition of claim 1, wherein greater than 90 mole percent of said plurality of N-glycans consists essentially of GlcNAcMan₃GlcNAc₂.

5: The composition of claim 1, wherein the GlcNAcMan₃GlcNAc₂ N-glycan is present at a level from about 5 mole percent to about 50 mole percent more than the next most predominant N-glycan structure of said plurality of N-glycans.

6: The composition of claim 1, wherein the immunoglobulins or fragments exhibit decreased binding affinity for an FcγRII receptor.

7: The composition of claim 6, wherein the FcγRII receptor is a FcγRIIa receptor.

8: The composition of claim 7, wherein the immunoglobulins or fragments exhibit decreased phagocytosis (clearance of immunocomplexes by macrophages).

9: The composition of claim 6, wherein the FcγRII receptor is a FcγRIIb receptor.

10: The composition of claim 9, wherein the immunoglobulins or fragments activate B cells.

11: The composition of claim 1, wherein the immunoglobulins or fragments exhibit increased binding affinity for an FcγRIII receptor.

12: The composition of claim 11, wherein the FcγRIII receptor is a FcγRIIIa receptor.

13: The composition of claim 11, wherein the FcγRIII receptor is a FcγRIIIb receptor.

14: The composition of claim 1, wherein the immunoglobulins or fragments exhibit increased antibody-dependent cellular cytotoxicity (ADCC) activity.

15: The composition of claim 1, wherein the immunoglobulins or fragments are essentially free of fucose.

16: The composition of claim 1, wherein the immunoglobulins or fragments lack fucose.

17: The composition of claim 1, wherein the immunoglobulins or fragments bind to an antigen selected from the group consisting of growth factors, FGFR, EGFR, VEGF, leukocyte antigens, CD20, CD33, cytokines, TNF-α, and TNF-β.

18: The composition of claim 1, wherein the immunoglobulins or fragments comprise an Fc region selected from the group consisting of an IgG1, IgG2, IgG3, and IgG4 Fc regions.

19-23. (canceled)

24: A kit comprising the composition of claim 1.

25: A eukaryotic host cell comprising an exogenous gene encoding an immunoglobulin or fragment thereof, wherein the eukaryotic host cell is genetically modified or selected to express the immunoglobulin composition of claim 1.

26. (canceled)

27: A method for producing a composition comprising a plurality of immunoglobulins or fragments, each immunoglobulin or fragment comprising at least one N-glycan attached thereto wherein the composition thereby comprises a plurality of N-glycans in which the predominant N-glycan consists essentially of GlcNAcMan₃GlcNAc₂ comprising:
(a) providing the eukaryote host cell of claim 25;
(b) growing the eukaryote host cell in a culture medium for a time sufficient for the eukaryote host cell to produce the immunoglobulins or fragments; and,
(c) isolating the immunoglobulins or fragments to produce the composition.

28. (canceled)
